SEQUENCE LISTING (2 sequences)

SEQ ID NO: 1 - DNA, 100 bp, Artificial Sequence
  Primer
cggccaagcttactcggatccagagtttgcaaaatatacaggggattatatataatggaaaacaagaaaggaaaataggaggtttatatggaagacgcca

SEQ ID NO: 2 - DNA, 20 bp, Artificial Sequence
  Primer
gtcatcgtcgggaagacctg

(19) United States
(12) Patent Application Publication
(10) Pub. No.: US 2008/0300231 A1
(43) Pub. Date: Dec. 4, 2008

(54) Antibacterial amide macrocycles VI

(76) Inventors: Rainer Endermann, Wuppertal (DE); Kerstin Ehlert, Velbert (DE); Siegfried Raddatz, Koeln (DE); Martin Michels, Koeln (DE); Yolanda Cancho-Grande, Leverkusen (DE); Stefan Weigand, Penzberg (DE); Karin Fischer, Solingen (DE)

Correspondence Address: MORRISON & FOERSTER LLP, 12531 HIGH BLUFF DRIVE, SUITE 100, SAN DIEGO, CA, 92130-2040, US

(21) Appl. No.: 11/904,550

(22) Filed: Sep. 26, 2007

Related U.S. Application Data
(63) Continuation of application No. PCT/EP2006/002564, filed on Mar. 21, 2006.

(30) Foreign Application Priority Data
Mar. 30, 2005 (DE) 10 2005 014 247.8

(51) Int. Cl. A61K 31/33; C07D 245/00; A61P 31/00

(52) U.S. Cl. 514/183; 540/460

(57) ABSTRACT

The invention relates to antibacterial amide macrocycles and methods for their preparation, their use for the treatment and/or prophylaxis of diseases, as well as their use for the production of medicaments for the treatment and/or prophylaxis of diseases, in particular of bacterial infections.

### CROSS REFERENCE TO RELATED APPLICATIONS

This application is a continuation of pending international application PCT/EP2006/002564, filed Mar. 21, 2006, designating US, which claims priority from German patent application DE 10 2005 014 247.8, filed Mar. 30, 2005. The contents of the above-referenced applications are incorporated herein by this reference in their entirety.

### BACKGROUND OF THE INVENTION

The invention relates to antibacterial amide macrocycles and methods for their preparation, their use for the treatment and/or prophylaxis of diseases, as well as their use for the production of medicaments for the treatment and/or prophylaxis of diseases, in particular of bacterial infections.

WO 03/106480, WO 04/012816, WO 05/033129, WO 05/058943, WO 05/100380 and WO 05/118613 describe macrocycles of the biphenomycin B type which have antibacterial activity and have amide or ester substituents respectively.

U.S. Pat. No. 3,452,136, thesis of R. U. Meyer, Stuttgart University, Germany 1991, thesis of V. Leitenberger, Stuttgart University, Germany 1991, Synthesis (1992), (10), 1025-30, J. Chem. Soc., Perkin Trans. 1 (1992), (1), 123-30, J. Chem. Soc., Chem. Commun. (1991), (10), 744, Synthesis (1991), (5), 409-13, J. Chem. Soc., Chem. Commun. (1991), (5), 275-7, J. Antibiot. (1985), 38(11), 1462-8, J. Antibiot. (1985), 38(11), 1453-61 describe the natural product biphenomycin B as having antibacterial activity. Some steps in the synthesis of biphenomycin B are described in Synlett (2003), 4, 522-526.

Chirality (1995), 7(4), 181-92, J. Antibiot. (1991), 44(6), 674-7, J. Am. Chem. Soc. (1989), 111(19), 7323-7, J. Am. Chem. Soc. (1989), 111(19), 7328-33, J. Org. Chem. (1987), 52(24), 5435-7, Anal. Biochem. (1987), 165(1), 108-13, J. Org. Chem. (1985), 50(8), 1341-2, J. Antibiot. (1993), 46(3), C-2, J. Antibiot. (1993), 46(1), 135-40, Synthesis (1992), (12), 1248-54, Appl. Environ. Microbiol. (1992), 58(12), 3879-8, J. Chem. Soc., Chem. Commun. (1992), (13), 951-3 describe a structurally related natural product, biphenomycin A, which has a further substitution with a hydroxy group on the macrocycle.

The natural products do not comply in terms of their properties with the requirements for antibacterial medicaments. Although structurally different agents with antibacterial activity are available on the market, the development of a resistance is a regular possibility. Novel agents for a good and more effective therapy are therefore desirable.

### SUMMARY OF THE INVENTION

One object of the present invention is therefore to provide novel and alternative compounds with the same or improved antibacterial activity for the treatment of bacterial diseases in humans and animals.

It has surprisingly been found that certain derivatives of these natural products in which the carboxy group of the natural product is replaced with a tertiary amide group which comprises a basic group have antibacterial activity against biphenomycin-resistant S. aureus strains (RN4220Bi^{R} and T17).

Furthermore, the derivatives show an improved spontaneous resistance rate for S. aureus wild-type strains and biphenomycin-resistant S. aureus strains.

The invention relates to compounds of formula

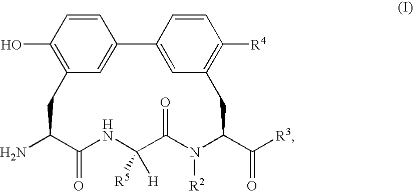

in which

R⁵ represents a group of formula

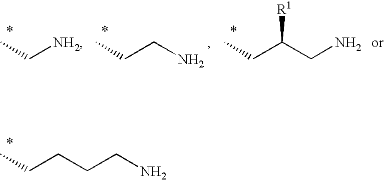

whereby

* is the linkage site to the carbon atom,

R¹ represents hydrogen or hydroxy,

R² represents hydrogen, methyl or ethyl,

R³ represents a group of formula

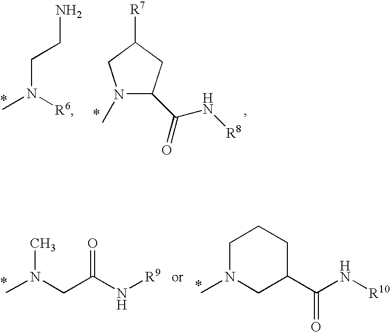

whereby

is the linkage site to the nitrogen atom,

R⁶ represents a group of formula

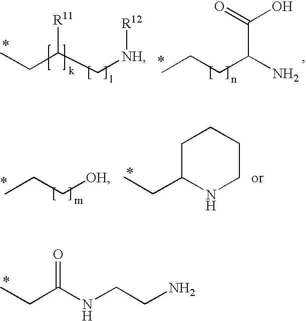

in which
* is the linkage site to the nitrogen atom,
R¹¹ represents hydrogen, amino or hydroxy,
R² represents hydrogen or methyl,
k is a number 0 or 1,
l is a number 1, 2, 3 or 4,
and
m and n independently of one another are a number 1, 2 or 3,

R⁷ represents hydrogen, amino or hydroxy,

R⁸, R⁹ and R¹⁰ independently of one another represent a group of formula

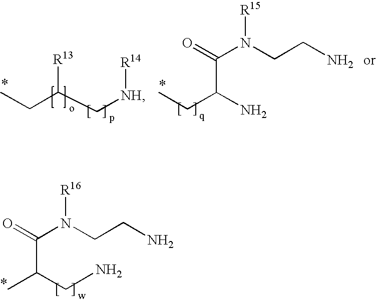

in which
* is the linkage site to the nitrogen atom
R¹³ represents hydrogen, amino or hydroxy,
R¹⁴ represents hydrogen or methyl,

R¹⁵ and R¹⁶ independently of one another represent hydrogen, aminoethyl or hydroxyethyl,

o is a number 0 or 1,

p, q and w independently of one another are a number 1, 2, 3 or 4,

R⁴ represents hydrogen, hydroxy, halogen, amino or methyl,

and their salts, their solvates and the solvates of their salts.

Compounds of the invention are the compounds of formula (I) and their salts, solvates and solvates of the salts, as well as the compounds which are encompassed by formula (I) and are mentioned below as exemplary embodiment(s), and their salts, solvates and solvates of the salts, insofar as the compounds which are encompassed by formula (I) and are mentioned below are not already salts, solvates and solvates of the salts.

The compounds of the invention may, depending on their structure, exist in stereoisomeric forms (enantiomers, diastereomers). The invention therefore relates to the enantiomers or diastereomers and respective mixtures thereof. The stereoisomerically pure constituents can be isolated in a known way from such mixtures of enantiomers and/or diastereomers by known methods such as chromatography on a chiral phase or crystallization using chiral amines or chiral acids.

The invention also relates, depending on the structure of the compounds, to tautomers of the compounds.

Salts preferred for the purposes of the invention are physiologically acceptable salts of the compounds of the invention.

Physiologically acceptable salts of the compounds (I) include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, malic acid, citric acid, fumaric acid, maleic acid, trifluoroacetic acid and benzoic acid.

Physiologically acceptable salts of the compounds (I) also include salts of usual bases such as, by way of example and preferably, alkali metal salts (e.g. sodium and potassium salts), alkaline earth metal salts (e.g. calcium and magnesium salts) and ammonium salts derived from ammonia or organic amines having 1 to 16 carbon atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, dihydroabietylamine, arginine, lysine, ethylenediamine and methylpiperidine.

Solvates for the purposes of the invention refer to those forms of the compounds which form a complex in the solid or liquid state through coordination with solvent molecules. Hydrates are a special form of solvates in which coordination takes place with water.

Halogen represents fluorine, chlorine, bromine and iodine.

A symbol # on a carbon atom means that the compound is, in terms of the configuration at this carbon atom, in enantiopure form, by which is meant for the purpose of the present invention an enantiomeric excess of more than 90% (>90% ee).

In the formulae of the groups standing for R³, the end point of the line besides which there is in each case an * does not represent a carbon atom or a CH₂ group but forms part of the bond to the carbonyl group to which R³ is bonded.

In the formulae of the groups which R⁵ can represent, the end point of the line beside which there is in each case an * does not represent a carbon atom or a CH₂ group but forms part of the bond to the carbon atom to which R⁷ is bonded.

In the formulae of the groups standing for R⁶, R⁸, R⁹ and R¹⁰, the end point of the line beside which there is in each case an * does not represent a carbon atom or a CH₂ group but forms part of the bond to the nitrogen atom to which R⁶, R⁸, R⁹ and R¹⁰, are bonded.

For the purpose of the present invention preference is also given to compounds of formula

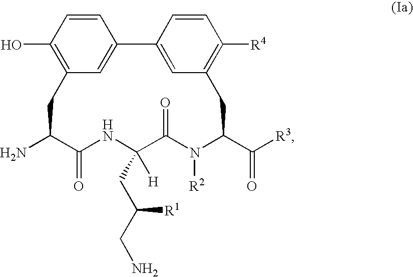

in which

R¹ represents hydrogen or hydroxy,

R² represents hydrogen, methyl or ethyl,

R³ is as defined above,

R⁴ represents hydrogen, hydroxy, halogen, amino or methyl,

and their salts, their solvates and the solvates of their salts.

For the purpose of the present invention preference is also given to compounds of formula (I) or (Ia) in which

R² represents hydrogen.

For the purpose of the present invention preference is also given to compounds of formula (I) or (Ia) in which

R⁴ represents hydrogen, hydroxy, chlorine or methyl.

For the purpose of the present invention preference is also given to compounds of formula (I) or (Ia) in which

R⁴ represents hydroxy.

For the purpose of the present invention preference is also given to compounds of formula (I) or (Ia) in which

R⁵ represents a group of formula

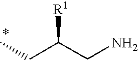

whereby

is the linkage site to the carbon atom,

R¹ represents hydrogen or hydroxy

For the purpose of the present invention preference is also given to compounds of formula (I) or (Ia) in which represents a group of formula

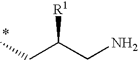

whereby

* is the linkage site to the carbon atom,

R¹ represents hydrogen or hydroxy,

R² represents hydrogen,

R³ represents a group of formula

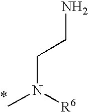

whereby

* is the linkage site to the nitrogen atom,

R⁶ represents a group of formula

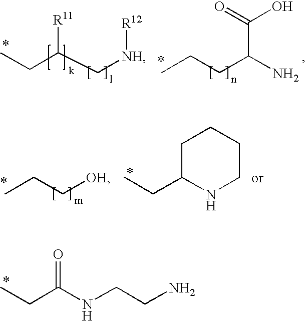

in which
* is the linkage site to the nitrogen atom,
R¹¹ represents hydrogen, amino or hydroxy,
R¹² represents hydrogen or methyl,
k is a number 0 or 1,
l is a number 1, 2, 3 or 4,
and
m and n independently of one another are a number 1, 2 or 3,

R⁴ represents hydroxy,

and their salts, their solvates and the solvates of their salts.

For the purpose of the present invention preference is also given to compounds of formula (I) or (Ia) in which

R⁵ represents a group of formula

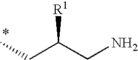

whereby

* is the linkage site to the carbon atom,

R¹ represents hydrogen or hydroxy,

R² represents hydrogen,

R³ represents a group of formula

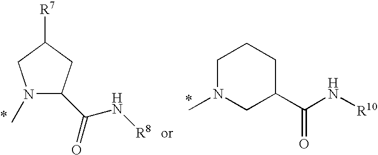

whereby

* is the linkage site to the nitrogen atom,

R⁷ represents hydrogen, amino or hydroxy,

R⁸ and R¹⁰ independently of one another represent a group of formula

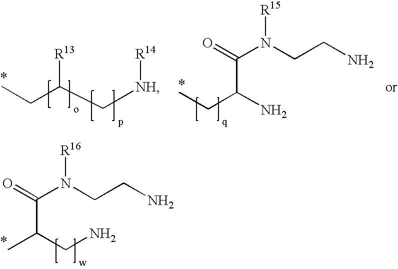

in which
* is the linkage site to the nitrogen atom,
R¹³ represents hydrogen, amino or hydroxy,
R¹⁴ represents hydrogen or methyl,
R¹⁵ and R¹⁶ independently of one another represent hydrogen, aminoethyl or hydroxyethyl,
o is a number 0 or 1,
p, q and w independently of one another are a number 1, 2, 3 or 4,

R⁴ represents hydroxy,

and their salts, their solvates and the solvates of their salts.

For the purpose of the present invention preference is also given to compounds of formula (I) or (Ia) in which

R⁵ represents a group of formula

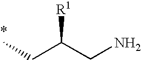

whereby

* is the linkage site to the carbon atom,

R¹ represents hydrogen or hydroxy,

R² represents hydrogen,

R³ represents a group of formula

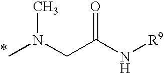

whereby

* is the linkage site to the nitrogen atom,

R⁹ represents a group of formula

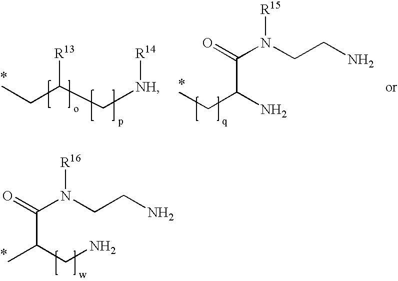

in which
* is the linkage site to the nitrogen atom,
R¹³ represents hydrogen, amino or hydroxy,
R¹⁴ represents hydrogen or methyl,
R¹⁵ and R¹⁶ independently of one another represent hydrogen, aminoethyl or hydroxyethyl,
o is a number 0 or 1,
p, q and w independently of one another are a number 1, 2, 3 or 4,

R⁴ represents hydroxy,

and their salts, their solvates and the solvates of their salts.

The invention further relates to a method for preparing the compounds of formula (I) or their salts, their solvates or the solvates of their salts, whereby according to method

[A] compounds of formula

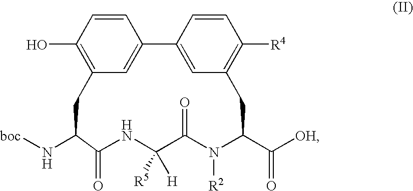

in which R², R⁴ and R⁵ have the abovementioned meaning, and boc represents tert-butoxycarbonyl,

are reacted in a two-stage process firstly in the presence of one or more dehydrating reagents with compounds of formula

HR³ (III)

in which R³ has the abovementioned meaning,

and subsequently with an acid and/or by hydrogenolysis,

or

[B] compounds of formula

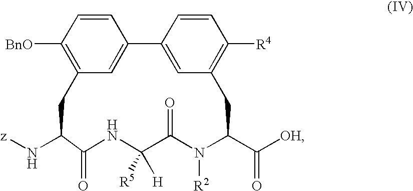

in which R², R⁴ and R⁵ have the abovementioned meaning, and Z represent benzyloxycarbonyl,

are reacted in a two-stage process firstly in the presence of one or more dehydrating reagents with compounds of formula

HR³ (III)

in which R³ has the abovementioned meaning,

and subsequently with an acid or by hydrogenolysis.

The free base of the salts can be obtained for example by chromatography on a reversed phase column with an acetonitrile-water gradient with the addition of a base, in particular by using an RP18 Phenomenex Luna C18(2) column and diethylamine as base.

The invention further relates to a method for preparing the compounds of formula (I) or their solvates according to claim 1 in which salts of the compounds or solvates of the salts of the compounds are converted into the compounds by chromatography with the addition of a base.

The hydroxy group on R¹ is, where appropriate, protected with a tert-butyldimethylsilyl group during the reaction with compounds of formula (III) which group is removed in the second reaction step.

Reactive functionalities in the radical R³ of compounds of formula (III) are introduced into the synthesis already protected, with preference for acid-labile protecting groups (e.g. boc). After reaction has taken place to give compounds of formula (I), the protecting groups can be removed by a deprotection reaction. This takes place by standard methods of protecting group chemistry. Deprotection reactions under acidic conditions or by hydrogenolysis are preferred.

The reaction in the first stage of methods [A] and [B] generally takes place in inert solvents, where appropriate in the presence of a base, preferably in a temperature range from 0° C. to 40° C. under atmospheric pressure.

Dehydrating reagents suitable hereby are for example carbodiimides such as, for example, N,N′-diethyl-, N,N′-dipropyl-, N,N′-diisopropyl-, N,N′-dicyclohexylcarbodiimide, N-(3-dimethylaminoisopropyl)-N′-ethylcarbodiimide hydrochloride (EDC), N-cyclohexylcarbodiimide-N′-propyloxymethyl-polystyrene (PS-carbodiimide) or carbonyl compounds such as carbonyldiimidazole, or 1,2-oxazolium compounds such as 2-ethyl-5-phenyl-1,2-oxazolium-3-sulfate or 2-tert-butyl-5-methylisoxazolium perchlorate, or acylamino compounds such as 2-ethoxy-1-ethoxycarbonyl-1,2-dihydroquinoline, or propanephosphonic anhydride, or isobutyl chloroformate, or bis(2-oxo-3-oxazolidinyl)phosphoryl chloride or benzotriazolyloxytri(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)phosphonium hexafluorophosphate (BOP), or benzotriazol-1-yloxytris(pyrrolidino)phosphonium hexafluorophosphate (PyBOP) or mixtures thereof, or mixtures thereof together with bases.

Examples of bases are alkali metal carbonates such as, for example, sodium or potassium carbonate, or bicarbonate, or organic bases such as trialkylamines, e.g. triethylamine, N-methylmorpholine, N-methylpiperidine, 4-dimethylaminopyridine or diisopropylethylamine.

The condensation is preferably carried out with HATU in the presence of a base, in particular diisopropylethylamine, or with PyBOP in the presence of a base, in particular diisopropylethylamine.

Examples of inert solvents are halohydrocarbons such as dichloromethane or trichloromethane, hydrocarbon such as benzene, or nitromethane, dioxane, dimethylformamide or acetonitrile. It is likewise possible to employ mixtures of the solvents. Dimethylformamide is particularly preferred.

The reaction with an acid in the second stage of methods [A] and [B] preferably takes place in a temperature range from 0° C. to 40° C. under atmospheric pressure.

Acids suitable hereby are hydrogen chloride in dioxane, hydrogen bromide in acetic acid or trifluoroacetic acid in methylene chloride.

The hydrogenolysis in the second stage of method [B] generally takes place in a solvent in the presence of hydrogen and palladium on activated carbon, preferably in a temperature range from 0° C. to 40° C. under atmospheric pressure.

Examples of solvents are alcohols such as methanol, ethanol, n-propanol or isopropanol, in a mixture with water and glacial acetic acid, with preference for a mixture of ethanol, water and glacial acetic acid.

The compounds of formula (III) are known or can be prepared in analogy to known methods.

The compounds of formula (II) are known or can be prepared by reacting compounds of formula

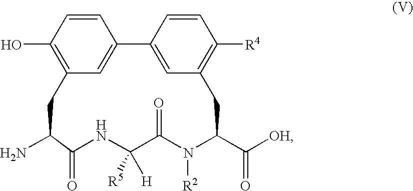

in which R², R⁴ and R⁵ have the abovementioned meaning,

with di(tert-butyl)dicarbonate in the presence of a base.

The reaction generally takes place in a solvent, preferably in a temperature range from 0° C. to 40° C. under atmospheric pressure.

Examples of bases are alkali metal hydroxides such as sodium or potassium hydroxide, or alkali metal carbonates such as cesium carbonate, sodium or potassium carbonate, or other bases such as DBU, triethylamine or diisopropylethylamine, with preference for sodium hydroxide or sodium carbonate.

Examples of solvents are halohydrocarbons such as methylene chloride or 1,2-dichloroethane, alcohols such as methanol, ethanol or isopropanol, or water.

The reaction is preferably carried out with sodium hydroxide in water or sodium carbonate in methanol.

The compounds of formula (V) are known or can be prepared by reacting compounds of formula

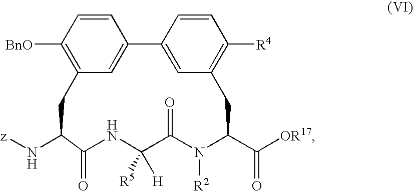

in which R², R⁴ and R⁵ have the abovementioned meaning, and

R¹⁷ represents benzyl, methyl or ethyl,

with an acid or by hydrogenolysis as described for the second stage of method [B], where appropriate by subsequent reaction with a base to hydrolyse the methyl or ethyl ester.

The hydrolysis can take place for example as described for the reaction of compounds of formula (VI) to give compounds of formula (IV).

The compounds of formula (IV) are known or can be prepared by hydrolysing the benzyl, methyl or ethyl ester in compounds of formula (VI).

The reaction generally takes place in a solvent in the presence of a base, preferably in a temperature range from 0° C. to 40° C. under atmospheric pressure.

Examples of bases are alkali metal hydroxides such as lithium, sodium or potassium hydroxide, with preference for lithium hydroxide.

Examples of solvents are halohydrocarbons such as dichloromethane or trichloromethane, ethers, such as tetrahydrofuran or dioxane, or alcohols such as methanol, ethanol or isopropanol, or dimethylformamide. It is likewise possible to employ mixtures of the solvents or mixtures of the solvents with water. Tetrahydrofuran or a mixture of methanol and water are particularly preferred.

The compounds of formula (VI) are known or can be prepared by reacting compounds of formula

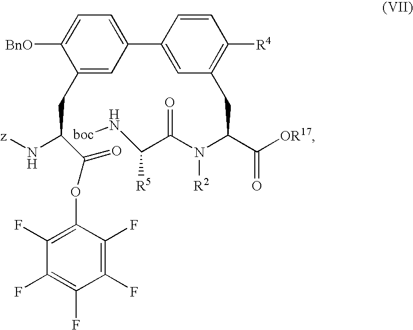

in which R², R⁴, R⁵ and R¹⁷ have the abovementioned meaning,

in the first stage with acids as described for the second stage of methods [A] and [B], and in the second stage with bases.

In the second stage the reaction with bases generally takes place in a solvent, preferably in a temperature range from 0° C. to 40° C. under atmospheric pressure.

Examples of bases are alkali metal hydroxides such as sodium or potassium hydroxide, or alkali metal carbonates such as cesium carbonate, sodium or potassium carbonate, or other bases such as DBU, triethylamine or diisopropylethylamine, with preference for triethylamine.

Examples of solvents are halohydrocarbons such as chloroform, methylene chloride or 1,2-dichloroethane, or tetrahydrofuran, or mixtures of the solvents, with preference for methylene chloride or tetrahydrofuran.

The compounds of formula (VII) are known or can be prepared by reacting compounds of formula

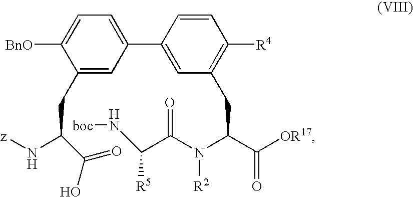

in which R², R⁴, R⁵ and R¹⁷ have the abovementioned meaning,

with pentafluorophenol in the presence of dehydrating reagents as described for the first stage of methods [A] and [B].

The reaction preferably takes place with DMAP and EDC in dichloromethane in a temperature range from −40° C. to 40° C. under atmospheric pressure.

The compounds of formula (VIII) are known or can be prepared by reacting compounds of formula

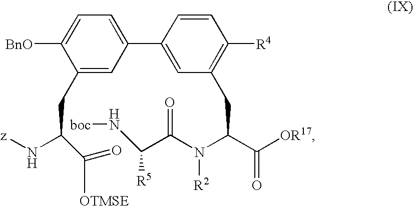

in which R², R⁴, R⁵ and R¹⁷ have the abovementioned meaning,

with fluoride, in particular with tetrabutylammonium fluoride.

The reaction generally takes place in a solvent, preferably in a temperature range from −10° C. to 30° C. under atmospheric pressure.

Examples of inert solvents are halohydrocarbons such as dichloromethane, or hydrocarbons such as benzene or toluene, or ethers such as tetrahydrofuran or dioxane, or dimethylformamide. It is likewise possible to employ mixtures of the solvents. Tetrahydrofuran and dimethylformamide are preferred solvents.

The compounds of formula (IX) are known or can be prepared by reacting compounds of formula

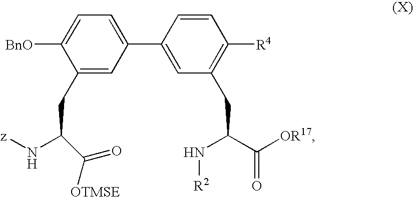

in which R², R⁴ and R¹⁷ have the abovementioned meaning,

with compounds of formula

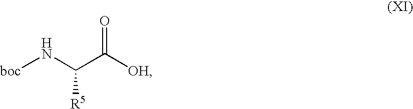

in which R⁵ has the abovementioned meaning,

in the presence of dehydrating reagents as described for the first stage of methods [A] and [B].

The compounds of formula (X) are known or can be prepared in analogy to the methods described in the examples section.

The compounds of formula (XI) are known or can be prepared in analogy to known methods.

The compounds of the invention show a valuable range of pharmacological and pharmacokinetic effects which could not have been predicted.

They are therefore suitable for use as medicaments for the treatment and/or prophylaxis of diseases in humans and animals.

The compounds of the invention can, because of their pharmacological properties, be employed alone or in combination with other active compounds for the treatment and/or prophylaxis of infectious diseases, especially of bacterial infections.

It is for example possible to treat and/or prevent local and/or systemic diseases caused by the following pathogens or by mixtures of the following pathogens:

gram-positive cocci, e.g. staphylococci (Staph. aureus, Staph. epidermidis) and streptococci (Strept. agalactiae, Strept. faecalis, Strept. pneumoniae, Strept. pyogenes); gram-negative cocci (neisseria gonorrhoeae) as well as gram-negative rods such as enterobacteriaceae, e.g. Escherichia coli, Haemophilus influenzae, Citrobacter (Citrob. freundii, Citrob. divernis), Salmonella and Shigella; furthermore klebsiellas (Klebs. pneumoniae, Klebs. oxytocy), Enterobacter (Ent. aerogenes, Ent. agglomerans), Hafnia, Serratia (Serr. marcescens), Proteus (Pr. mirabilis, Pr. rettgeri, Pr. vulgaris), Providencia, Yersinia, and the genus Acinetobacter. The antibacterial range additionally includes the genus Pseudomonas (Ps. aeruginosa, Ps. maltophilia) as well as strictly anaerobic bacteria such as Bacteroides fragilis, representatives of the genus Peptococcus, Peptostreptococcus, as well as the genus Clostridium; furthermore mycoplasmas (M. pneumoniae, M. hominis, M. urealyticum) as well as mycobacteria, e.g. Mycobacterium tuberculosis.

The above list of pathogens is merely by way of example and is by no means to be interpreted restrictively. Examples which may be mentioned of diseases which are caused by the pathogens mentioned or mixed infections and can be prevented, improved or healed by the topically applicable preparations of the invention, are:

infectious diseases in humans such as, for example, septic infections, bone and joint infections, skin infections, postoperative wound infections, abscesses, phlegmon, wound infections, infected burns, burn wounds, infections in the oral region, infections after dental operations, septic arthritis, mastitis, tonsillitis, genital infections and eye infections.

Apart from humans, bacterial infections can also be treated in other species. Examples which may be mentioned are:

Pigs: coli diarrhea, enterotoxemia, sepsis, dysentery, salmonellosis, metritis-mastitis-agalactiae syndrome, mastitis;

Ruminants (cattle, sheep, goats): diarrhea, sepsis, bronchopneumonia, salmonellosis, pasteurellosis, mycoplasmosis, genital infections;

Horses: bronchopneumonias, joint ill, puerperal and postpuerperal infections, salmonellosis;

Dogs and cats: bronchopneumonia, diarrhea, dermatitis, otitis, urinary tract infections, prostatitis;

Poultry (chickens, turkeys, quail, pigeons, ornamental birds and others): mycoplasmosis, E. coli infections, chronic airway diseases, salmonellosis, pasteurellosis, psittacosis.

It is likewise possible to treat bacterial diseases in the rearing and management of productive and ornamental fish, in which case the antibacterial spectrum is extended beyond the pathogens mentioned above to further pathogens such as, for example, Pasteurella, Brucella, Campylobacter, Listeria, Erysipelothris, corynebacteria, Borellia, Treponema, Nocardia, Rikettsie, Yersinia.

The present invention further relates to the use of the compounds of the invention for the treatment and/or prophylaxis of diseases, preferably of bacterial diseases, especially of bacterial infections.

The present invention further relates to the use of the compounds of the invention for the treatment and/or prophylaxis of diseases, especially of the aforementioned diseases.

The present invention further relates to the use of the compounds of the invention for the production of a medicament for the treatment and/or prophylaxis of diseases, especially of the aforementioned diseases.

The present invention further relates to a method for the treatment and/or prophylaxis of diseases, especially of the aforementioned diseases, using an antibacterially effective amount of the compounds of the invention.

The compounds of the invention may act systemically and/or locally. For this purpose, they can be administered in a suitable way such as, for example, orally, parenterally pulmonarily, nasally, sublingually, lingually, buccally, rectally, dermally, transdermally, conjunctivally or optically or as an implant or stent.

The compounds of the invention can be administered in administration forms suitable for these administration routes.

Suitable for oral administration are administration forms which function according to the prior art and deliver the compounds of the invention rapidly and/or in modified fashion, and which contain the compounds of the invention in crystalline and/or amorphized and/or dissolved form, such as, for example, tablets (uncoated or coated tablets, for example having coatings which are resistant to gastric juice or dissolve with a delay or are insoluble and control the release of the compound of the invention), tablets or films/wafers which disintegrate rapidly in the oral cavity, films/lyophilisates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration are, inter alia, preparations for injection and infusion in the form of solutions, suspensions, emulsions, lyophilisates or sterile powders.

Suitable for the other administration routes are, for example, pharmaceutical forms for inhalation (inter alia powder inhalers, nebulizers), nasal drops, solutions, sprays; tablets, films/wafers or capsules, for lingual, sublingual or buccal administration, suppositories, preparations for the ears or eyes, vaginal capsules, aqueous suspensions (lotions, shaking mixtures), lipophilic suspensions, ointments, creams, transdermal therapeutic systems (such as, for example, patches), milk, pastes, foams, dusting powders, implants or stents.

The compounds of the invention can be converted into the stated administration forms. This can take place in a manner known per se by mixing with inert, nontoxic, pharmaceutically acceptable excipients. These excipients include, inter alia, carriers (for example microcrystalline cellulose, lactose, mannitol), solvents (e.g. liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g. antioxidants such as, for example, ascorbic acid), colors (e.g. inorganic pigments such as, for example, iron oxides) and taste and/or odor corrigents.

The present invention further relates to medicaments which comprise at least one compound of the invention, usually together with one or more inert, nontoxic, pharmaceutically acceptable excipients, and to the use thereof for the aforementioned purposes.

It has generally proved advantageous on parenteral administration to administer amounts of about 5 to 250 mg/kg of body weight per 24 h to achieve effective results. The amount on oral administration is about 5 to 100 mg/kg of body weight per 24 h.

It may nevertheless be necessary where appropriate to deviate from the stated amounts, in particular as a function of body weight, administration route, individual behavior towards the active compound, nature of the preparation and time or interval over which the administration takes place. Thus, it may be sufficient in some cases to make do with less than the aforementioned minimum amount, whereas in other cases the stated upper limit must be exceeded. In case of an administration of larger amounts, it may be advisable to divide these into a plurality of single doses over the day.

The percentage data in the following tests and examples are percentages by weight unless otherwise indicated; parts are parts by weight. Solvent ratios, dilution ratios and concentration data for liquid/liquid solutions are in each case based on volume.

### DETAILED DESCRIPTION OF PREFERRED EMBODIMENTS

### A. Examples

### Abbreviations Used

abs. absolute
aq. aqueous
Bn benzyl
boc tert-butoxycarbonyl
CDCl₃ chloroform
CH cyclohexane
d doublet (in ¹H NMR)
dd doublet of doublets (in ¹H NMR)
DCC dicyclohexylcarbodiimide
DIC diisopropylcarbodiimide
DIEA diisopropylethylamine (Hünig's base)
DMSO dimethyl sulfoxide

### DMAP 4-N,N-dimethylaminopyridine

DMF dimethylformamide
EA ethyl acetate (acetic acid ethyl ester)
EDC N′-(3-dimethylaminopropyl)-N-ethylcarbodiimide×HCl
ESI electrospray ionization (in MS)
Ex. example
Fmoc 9-fluorenylmethoxycarbonyl
HATU O-(7-azabenzotriazol-1-yl)-N,N,N′-tetramethyluronium hexafluorophosphate
HBTU O-(benzotriazol-1-yl)-N,N,N′,N′-tetramethyluronium hexafluorophosphate
HOBt 1-hydroxy-1H-benzotriazole×H₂O
h hour(s)
HPLC high pressure, high performance liquid chromatography
LC-MS coupled liquid chromatography-mass spectroscopy
m multiplet (in ¹H NMR)
min minute
MS mass spectroscopy
NMR nuclear magnetic resonance spectroscopy
MTBE methyl tert-butyl ether
Pd/C palladium/carbon
PFP pentafluorophenol
PyBOP benzotriazol-1-yloxytris(pyrrolidino)phosphonium hexafluorophosphate
q quartet (in ¹H NMR)
R_{f} retention index (in TLC)
RP reverse phase (in HPLC)
RT room temperature
Rₜ retention time (in HPLC)
s singlet (in ¹H NMR)
sat saturated
t triplet (in ¹H NMR)
TBS tert-butyldimethylsilyl
TFA trifluoroacetic acid
THF tetrahydrofuran
TLC thin-layer chromatography
TMSE 2-(trimethylsilyl)ethyl
TPTU 2-(2-oxo-1(2H)-pyridyl)-1,1,3,3,-tetramethyluronium tetrafluoroborate
Z benzyloxycarbonyl

LC-MS and HPLC Methods:

Method 1 (LC-MS): MS instrument type: Micromass ZQ; HPLC instrument type: Waters Alliance 2795; column: Phenomenex Synergi 2μ Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

Method 2 (LC-MS): MS instrument type: Micromass ZQ; HPLC instrument type: HP 1100 Series; UV DAD; column: Phenomenex Synergi 2μ Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

Method 3 (LC-MS): Instrument: Micromass Quattro LCZ with HPLC Agilent Series 1100; column: Phenomenex Synergi 2μ Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 208-400 nm.

Method 4 (LC-MS): MS instrument type: Micromass ZQ; HPLC instrument type: Waters Alliance 2795; column: Merck Chromolith SpeedROD RP-18e 50 mm×4.6 mm; eluent A: water+500 μl of 50% formic acid/l; eluent B: acetonitrile+500 μl of 50% formic acid/l; gradient: 0.0 min 10% B→3.0 min 95% B→4.0 min 95% B; oven: 35° C.; flow rate: 0.0 min 1.0 ml/min→3.0 min 3.0 ml/min→4.0 min 3.0 ml/min; UV detection: 210 nm.

Method 5 (LC-MS): Instrument: Micromass Platform LCZ with HPLC Agilent Series 1100; column: Thermo HyPURITY Aquastar 3μ50 mm×2.1 mm; eluent A: 1 l of water+0.5 ml of 50% formic acid, eluent B: 1 l of acetonitrile+0.5 ml of 50% formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

Method 6 (LC-MS): Instrument: Micromass Platform LCZ with HPLC Agilent Series 1100; column: Thermo Hypersil GOLD-3μ20 mm×4 mm; eluent A: 1 l of water+0.5 ml of 50% formic acid, eluent B: 1 l of acetonitrile+0.5 ml of 50% formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

Starting Compounds

### Example 1A

Benzyl{(1S)-4-[(tert-butoxycarbonyl)amino]-1-[({2-[(tert-butoxycarbonyl)amino]-ethyl}amino)carbonyl]butyl}carbamate

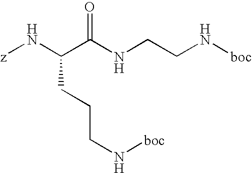

300 mg (0.82 mmol) of N²-[(benzyloxy)carbonyl]-N⁵-(tert-butoxycarbonyl)-L-ornithine and 171 mg (1.06 mmol) of tert-butyl-(2-aminoethyl)carbamate are dissolved in 6 ml of dimethylformamide under argon. Then, at 0° C. (ice bath), 204 mg (1.06 mmol) of EDC and 33 mg (0.25 mmol) of HOBt are added. The mixture is slowly warmed to RT and stirred at RT for 12 h. The solution is concentrated in vacuo and the residue is taken up with ethyl acetate. The organic phase is washed successively with a saturated sodium bicarbonate solution and a sodium chloride solution, dried over magnesium sulfate and evaporated in vacuo. The remaining solid is dried under high vacuum.

Yield: 392 mg (94% of theory)

LC-MS (method 1): Rₜ=2.36 min.

MS (ESI): m/z=509 (M+H)⁺

### Example 2A

### N⁵-(tert-Butoxycarbonyl)-N-{2-[(tert-butoxycarbonyl)amino]ethyl}-L-ornithinamide

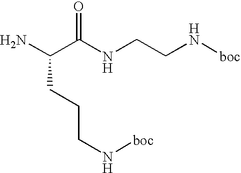

A solution of 390 mg (0.77 mmol) of benzyl{(1S)-4-[(tert-butoxycarbonyl)amino]-1-[({2-[(tert-butoxycarbonyl)amino]ethyl}amino)carbonyl]butyl}carbamate (Example 1A) in 50 ml of ethanol is hydrogenated after the addition of 40 mg of palladium on activated carbon (10%) at RT under atmospheric pressure for 4 h. The mixture is filtered through kieselguhr and the residue is washed with ethanol. The filtrate is evaporated to dryness in vacuo. The product is reacted without further purification.

Yield: 263 mg (91% of theory)

MS (ESI): m/z=375 (M+H)⁺; 397 (M+Na)⁺.

### Example 3A

### 1-[(Benzyloxy)carbonyl]-L-prolyl-N⁵-(tert-butoxycarbonyl)-N-{2-[(tert-butoxycarbonyl)amino]ethyl}-L-ornithinamide

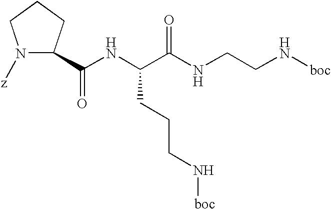

48 mg (0.194 mmol) of 1-[(benzyloxy)carbonyl]-L-proline and 94 mg (0.25 mmol) of the compound from Example 2A are dissolved in 6 ml of dimethylformamide under argon. Then, at 0° C. (ice bath), 48 mg (0.25 mmol) of EDC and 7.8 mg (0.058 mmol) of HOBt are added. The mixture is slowly warmed to RT and stirred at RT for 12 h. The solution is concentrated in vacuo and the residue is taken up in dichloromethane and washed with a saturated sodium bicarbonate solution, 0.1N hydrochloric acid and water. The combined organic phases are concentrated in vacuo, and the solid obtained in this way is reacted further without purification.

Yield: 117 mg (95% of theory)

LC-MS (method 3): Rₜ=2.36 min.

MS (ESI): m/z=606 (M+H)⁺

### Example 4A

### L-Prolyl-N⁵-(tert-butoxycarbonyl)-N-{2-[(tert-butoxycarbonyl)amino]ethyl}-L-ornithinamide

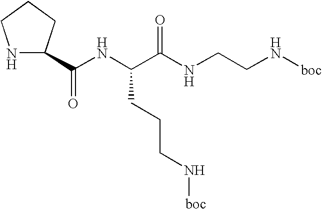

117 mg (0.193 mmol) of the compound from Example 3A are dissolved in 50 ml of ethanol, and 20 mg of palladium on activated carbon (10%) are added. Hydrogenation is carried out under atmospheric pressure for 12 h and, after filtration through kieselguhr, the filtrate is concentrated in vacuo. The solid obtained in this way is reacted further without purification.

Yield: 86 mg (94% of theory)

MS (ESI): m/z=472 (M+H)⁺

### Example 5A

### tert-Butyl{(5S)-5-[(tert-butoxycarbonyl)amino]-6-hydroxyhexyl}carbamate

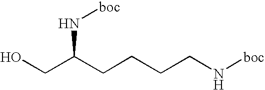

91 mg (0.90 mmol) of 4-methylmorpholine and 98 mg (0.90 mmol) of ethyl chloroformate are added to a solution of 475 mg (0.90 mmol) N²,N⁶-bis(tert-butoxycarbonyl)-L-lysine —N-cyclohexylcyclohexanamine (1:1) in 10 ml of tetrahydrofuran at −10° C., and the mixture is stirred for 30 min. At this temperature, 1.81 ml (1.81 mmol) of a 1M solution of lithium aluminum hydride in tetrahydrofuran are slowly added dropwise. The mixture is slowly warmed to RT and stirred at RT for 12 h. While cooling in ice, 0.1 ml of water and 0.15 ml of a 4.5% sodium hydroxide solution are cautiously added, and stirring is continued at RT for 3 h. The mixture is filtered and the filtrate is concentrated in vacuo. The residue is dissolved in ethyl acetate, washed with water, dried over magnesium sulfate and again evaporated to dryness in vacuo. The product is reacted without further purification.

Yield: 250 mg (83% of theory)

MS (ESI): m/z=333 (M+H)⁺

### Example 6A

### (2S)-2,6-Bis[(tert-butoxycarbonyl)amino]hexyl methanesulfonate

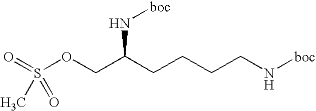

103 mg (0.90 mmol) of methanesulfonyl chloride and 0.21 ml (1.5 mmol) of triethylamine are added to a solution of 250 mg (0.75 mmol) of the compound from Example 5A in 20 ml of dichloromethane and the mixture is stirred at RT for 16 h. The mixture is diluted with dichloromethane and washed twice with 0.1N hydrochloric acid. The organic phase is dried over magnesium sulfate and evaporated to dryness in vacuo. The product is reacted without further purification.

Yield: 264 mg (86% of theory)

MS (DCI): m/z=428 (M+NH₄)⁺.

### Example 7A

### tert-Butyl{(5S)-6-azido-5-[(tert-butoxycarbonyl)amino]hexyl}carbamate

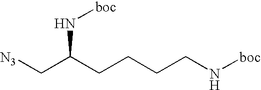

42 mg (0.64 mmol) of sodium azide are added to a solution of 264 mg (0.64 mmol) of the compound from Example 6A in 15 ml of dimethylformamide and the mixture is stirred at 70° C. for 12 h. Most of the solvent is distilled off in vacuo, and the residue is diluted with ethyl acetate. The mixture is washed several times with a saturated sodium bicarbonate solution, dried over magnesium sulfate and evaporated to dryness in vacuo. The product is reacted without further purification.

Yield: quant.

MS (ESI): m/z=358 (M+H)⁺.

### Example 8A

### tert-Butyl{(5S)-6-amino-5-[(tert-butoxycarbonyl)amino]hexyl}carbamate

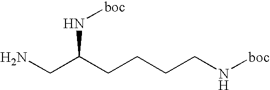

A solution of 229 mg (0.64 mmol) of the compound from Example 7A in 10 ml of ethanol is hydrogenated after the addition of 20 mg of palladium on activated carbon (10%) at RT under atmospheric pressure for 12 h. The mixture is filtered through kieselguhr, and the residue is washed with ethanol. The filtrate is evaporated to dryness in vacuo. The product is reacted without further purification.

Yield: 161 mg (76% of theory)

MS (ESI): m/z=332 (M+H)⁺.

### Example 9A

### Benzyl (2S,4R)-2-[({(2S)-2,6-bis[(tert-butoxycarbonyl)amino]hexyl}amino)carbonyl]-4-hydroxypyrrolidine-1-carboxylate

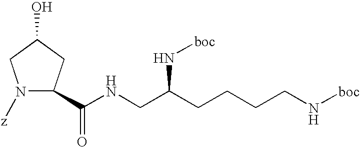

Preparation takes place in analogy to Example 3A from 57 mg (0.216 mmol) of (4R)-1-[(benzyloxy)carbonyl]-4-hydroxy-L-proline and 93 mg (0.28 mmol) of the compound from Example 8A in 6 ml of dimethylformamide with the addition of 54 mg (0.28 mmol) of EDC and 8.7 mg (0.065 mmol) of HOBt. The product is purified by preparative RP-HPLC (mobile phase water/acetonitrile Gradient: 90:10→5:95).

Yield: 42 mg (34% of theory)

LC-MS (method 1): Rₜ=2.08 min.

MS (ESI): m/z=579 (M+H)⁺

### Example 10A

### (4R)—N-{(2S)-2,6-bis[(tert-Butoxycarbonyl)amino]hexyl}-4-hydroxy-L-prolinamide

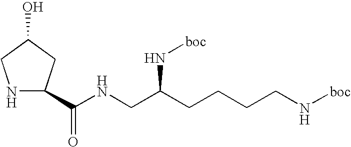

Preparation takes place in analogy to Example 4A from 41 mg (0.071 mmol) of the compound from Example 9A in 20 ml of ethanol with the addition of 7.5 mg of palladium on activated carbon (10%). The product is reacted without further purification.

Yield: quant.

MS (ESI): m/z=445 (M+H)⁺

### Example 11A

### 2-{[(Benzyloxy)carbonyl]amino}ethyl methanesulfonate

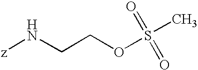

11.3 g (98.4 mmol) of methanesulfonyl chloride are added to a solution of 16 g (82.0 mmol) of benzyl (2-hydroxyethyl)carbamate and 16.60 g (64.02 mmol) of triethylamine in 1 l of dichloromethane. The reaction mixture is stirred at RT overnight. Water is added, and the organic phase is washed successively with water and a sodium chloride solution, dried over magnesium sulfate and evaporated in vacuo. The remaining solid is dried under high vacuum. The crude product is purified by preparative HPLC.

Yield 7 g (31% of theory)

LC-MS (method 3): Rₜ=1.84 min.

MS (ESI): m/z=273 (M+H)⁺.

### Example 12A

### Benzyl{2-[(2-hydroxyethyl)amino]ethyl}carbamate

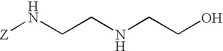

500 mg (1.83 mmol) of 2-{[(benzyloxy)carbonyl]amino}ethyl methanesulfonate (Example 11A) and 758 mg (5.48 mmol) of potassium carbonate are added to a solution of 226 mg (3.66 mmol) of 2-aminoethanol in 25 ml of acetonitrile. The reaction mixture is stirred at 50° C. overnight. The solvent is then evaporated and the residue is taken up in dichloromethane. The organic phase is washed with water, dried over magnesium sulfate and concentrated. The crude product is purified by preparative HPLC.

Yield 131 mg (29% of theory)

LC-MS (method 3): Rₜ=0.78 min.

MS (ESI): m/z=239 (M+H)⁺.

### Example 13A

### Benzyl[2-({2-[(tert-butoxycarbonyl)(methyl)amino]ethyl}amino)ethyl]carbamate

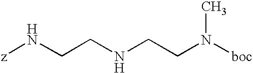

Preparation takes place in analogy to Example 12A from 300 mg (1.098 mmol) of 2-{[(benzyloxy)carbonyl]amino}ethyl methanesulfonate (Example 11A), 386 mg (2.19 mmol) of tert-butyl (2-aminoethyl)methylcarbamate and 455 mg (3.30 mmol) of potassium carbonate in 10 ml of acetonitrile.

Yield: 360 mg (82% of theory)

LC-MS (method 4): Rₜ=1.51 min.

MS (ESI): m/z=352 (M+H)⁺.

### Example 14A

Benzyl[2-({3-[(tert-butoxycarbonyl)amino]-2-hydroxypropyl}amino)ethyl]carbamate

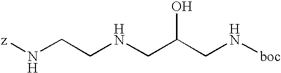

Preparation takes place in analogy to Example 12A from 270 mg (0.98 mmol) of 2-{[(benzyloxy)carbonyl]amino}ethyl methanesulfonate (Example 11A), 379 mg (1.98 mmol) of tert-butyl (3-amino-2-hydroxypropyl)carbamate and 409 mg (2.96 mmol) of potassium carbonate in 10 ml of acetonitrile.

Yield: 209 mg (45% of theory)

LC-MS (method 2): Rₜ=1.44 min.

MS (ESI): m/z=368 (M+H)⁺.

### Example 15A

### 1-tert-Butyl 5-(methoxycarbonyl) N-(tert-butoxycarbonyl)-L-glutamate

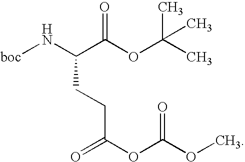

5 g (16.15 mmol) of (4S)-5-tert-butoxy-4-[(tert-butoxycarbonyl)amino]-5-pentanoic acid and 2.45 ml (17.60 mmol) of triethylamine are dissolved in 80 ml of THF under argon and cooled to 0° C. 1.68 g (17.77 mmol) of methyl chloroformate are added thereto, and the mixture is stirred at 0° C. for 3 hours. The reaction mixture is filtered through kieselguhr. The filtrate is reacted directly.

### Example 16A

### tert-Butyl N-(tert-butoxycarbonyl)-5-hydroxy-L-norvalinate

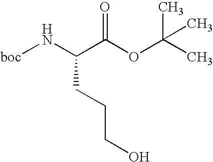

The filtrate of (1-tert-butyl 5-(methoxycarbonyl)-N-(tert-butoxycarbonyl)-L-glutamate (Example 15A) is added dropwise to a suspension of 1.52 g (40.38 mmol) of sodium borohydride in 4.5 ml of water at 0° C. The mixture slowly warms to RT and is stirred overnight. The reaction solution is concentrated in vacuo and, for working up, the residue is mixed with ethyl acetate and water. The organic phase is washed with a saturated sodium chloride solution, dried over magnesium sulfate and evaporated in vacuo. The crude product is reacted without further purification.

Yield: 4.5 g (48% of theory)

LC-MS (method 2): Rₜ=2.04 min.

MS (ESI): m/z=290 (M+H)⁺.

### Example 17A

### tert-Butyl N-(tert-butoxycarbonyl)-5-[(methylsulfonyl)oxy]-L-norvalinate

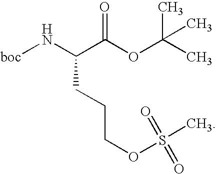

1.07 g (9.35 mmol) of methanesulfonyl chloride are added to a mixture of 4.5 g (7.79 mmol) of tert-butyl N-(tert-butoxycarbonyl)-5-hydroxy-L-norvalinate (Example 16A) and 2.17 ml (5.58 mmol) of triethylamine in 200 ml of dichloromethane. The mixture is stirred at RT overnight and then water is added. The organic phase is washed successively with water and a saturated sodium chloride solution, dried over magnesium sulfate and evaporated in vacuo. The crude product is purified by preparative HPLC.

Yield: quant.

LC-MS (method 1): Rₜ=2.16 min.

MS (ESI): m/z=368 (M+H)⁺.

### Example 18A

### tert-Butyl N²-(tert-butoxycarbonyl)-N⁵-{2-[(tert-butoxycarbonyl)amino]ethyl}-L-ornithinate

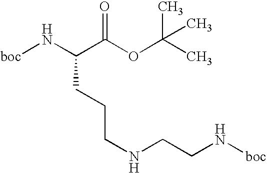

Preparation takes place in analogy to Example 12A from 2 g (5.443 mmol) of tert-butyl N-(tert-butoxycarbonyl)-5-[(methylsulfonyl)oxy]-L-norvalinate (Example 17A), 1.78 g (10.89 mmol) of tert-butyl (2-aminoethyl)carbamate and 2.26 g (16.33 mmol) of potassium carbonate in 100 ml of acetonitrile. The crude product is reacted without further purification.

Yield: 4.2 g (53% of theory)

LC-MS (method 3): Rₜ=1.61 min.

MS (ESI): m/z=432 (M+H)⁺.

### Example 19A

### tert-Butyl 2-{[(2-{[(benzyloxy)carbonyl]amino}ethyl)amino]methyl}piperidine-1-carboxylate

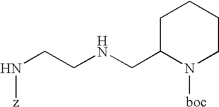

Preparation takes place in analogy to Example 12A from 1 g (3.66 mmol) of 2-{[(benzyloxy)carbonyl]amino}ethyl methanesulfonate (Example 11A), 1.56 g (7.31 mmol) of tert-butyl 2-(aminomethyl)piperidine-1-carboxylate and 1.52 g (10.98 mmol) of potassium carbonate in 70 ml of acetonitrile.

Yield: 680 mg (45% of theory)

LC-MS (method 1): Rₜ=1.47 min.

MS (ESI): m/z=392 (M+H)⁺.

### Example 20A

Benzyl{2-[{(2S)-5-{[(benzyloxy)carbonyl]amino}-2-[(tert-butoxycarbonyl)amino]pentanoyl}(2-hydroxyethyl)amino]ethyl}carbamate

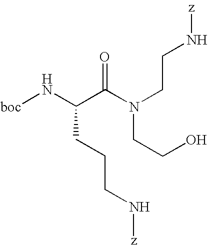

193 mg (0.51 mmol) of HATU and 0.247 ml (1.39 mmol) of N,N-diisopropylethylamine are added to a solution of 169 mg (0.462 mmol) of N⁵-[(benzyloxy)carbonyl]-N²-(tert-butoxycarbonyl)-L-ornithine in 10 ml of anhydrous DMF. After stirring at RT for 30 min, 116 mg (0.46 mmol) of benzyl{2-[(2-hydroxyethyl)amino]ethyl}carbamate (Example 12A) are added. The reaction mixture is stirred at RT for 15 h. The solvent is then evaporated and the residue is mixed in ethyl acetate and water. The organic phase is washed successively with 1N hydrochloric acid and a saturated aqueous sodium chloride solution, dried over sodium sulfate and concentrated. The crude product is purified by preparative HPLC.

Yield 188 mg (70% of theory)

LC-MS (method 1): Rₜ=2.17 min.

MS (ESI): m/z=587 (M+H)⁺.

### Example 21A

Benzyl{(4S)-4-amino-5-[(2-{[(benzyloxy)carbonyl]amino}ethyl)(2-hydroxyethyl)amino]-5-oxopentyl}carbamate hydrochloride

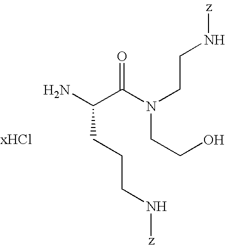

2 ml of a 4M hydrogen chloride solution in dioxane are added to a solution of 176 mg (0.30 mmol) of benzyl{2-[{(2S)-5-{[(benzyloxy)carbonyl]amino}-2-[(tert-butoxycarbonyl)amino]pentanoyl}(2-hydroxyethyl)amino]ethyl}carbamate (Example 20A) in 1 ml of dioxane. After 3 h at RT, the reaction solution is concentrated in vacuo, coevaporated with dichloromethane several times and dried under high vacuum. The crude product is reacted without further purification.

Yield: 160 mg (85% of theory)

LC-MS (method 1): Rₜ=1.53 min.

MS (ESI): m/z=487 (M−HCl+H)⁺.

### Example 22A

### tert-Butyl 3-[(3S)-3-(3-{[(benzyloxy)carbonyl]amino}propyl)-5-(2-hydroxyethyl)-4,9-dioxo-11-phenyl-10-oxa-2,5,8-triazaundecan-1-oyl]piperidine-1-carboxylate

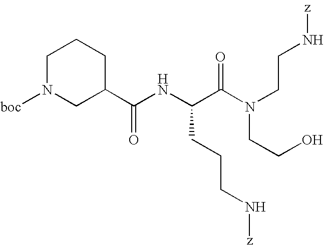

47 mg (0.206 mmol) of 1-(tert-butoxycarbonyl)piperidine-3-carboxylic acid, 130 mg (0.206 mmol) of benzyl{(4S)-4-amino-5-[(2-{[(benzyloxy)carbonyl]amino}ethyl)(2-hydroxyethyl)amino]-5-oxopentyl}carbamate hydrochloride (Example 21A) and 0.08 ml of triethylamine (0.56 mmol) are dissolved in 10 ml of dimethylformamide under argon. Then, at 0° C. (ice bath), 67 mg (0.350 mmol) of EDC and 9 mg (0.068 mmol) of HOBt are added. The mixture is slowly warmed to RT and stirred at RT for 12 h. The solution is concentrated in vacuo and the residue is taken up in dichloromethane. The organic phase is washed successively with water, 1N hydrochloric acid and a saturated aqueous sodium chloride solution, dried over sodium sulfate and concentrated. The crude product is reacted without further purification.

Yield: quant.

LC-MS (method 1): Rₜ=2.26 min.

MS (ESI): m/z=698 (M+H)⁺.

### Example 23A

Benzyl{(4S)-5-[(2-{[(benzyloxy)carbonyl]amino}ethyl)(2-hydroxyethyl)amino]-5-oxo-4-[(piperidin-3-ylcarbonyl)amino]pentyl}carbamate hydrochloride

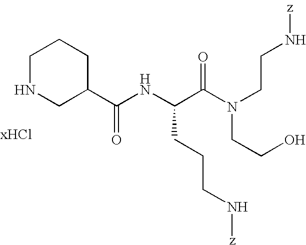

2 ml of a 4 M hydrogen chloride solution in dioxane are added to a solution of 180 mg (0.196 mmol) of tert-butyl 3-[(3S)-3-(3-{[(benzyloxy)carbonyl]amino}propyl)-5-(2-hydroxyethyl)-4,9-dioxo-11-phenyl-10-oxa-2,5,8-triazaundecan-1-oyl]-piperidine-1-carboxylate (Example 22A) in 1 ml of dioxane. After 3 h at RT, the reaction solution is concentrated in vacuo, coevaporated with dichloromethane several times and dried under high vacuum. The crude product is purified by preparative HPLC.

Yield: 18 mg (14% of theory)

LC-MS (method 2): Rₜ=1.84 min.

MS (ESI): m/z=598 (M−HCl+H)⁺.

Examples 24A to 29A listed in the following table are prepared from the corresponding starting materials in analogy to the method of Example 1A detailed above:

| Ex. | | | |
|---|---|---|---|
| No. | Structure | Prepared from | Analytical data |
| 24A | 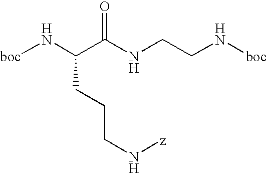 | N³-[(benzyloxy)-carbonyl]-N²-(tert-butoxycarbonyl)-L-ornithineand tert-butyl(2-aminoethyl)carbamate | LC-MS (method 3):Rₜ = 2.33 min.MS (ESI): m/z = 509(M + H)⁺ |
| 25A | 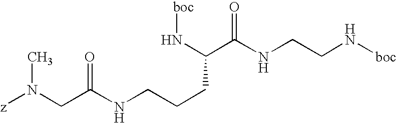 | N-[(benzyloxy)-carbonyl]-N-methylglycineand 30A | LC-MS (method 2):Rₜ = 2.26 min.MS (ESI): m/z = 579(M + H)⁺ |
| 26A | 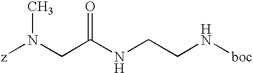 | N-[(benzyloxy)-carbonyl]-N-methylglycineand tert-butyl(2-aminoethyl)carbamate | LC-MS (method 3):Rₜ = 2.03 min.MS (ESI): m/z = 366(M + H)⁺ |
| 27A | 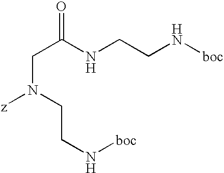 | N-[(benzyloxy)carbonyl]-N- {2-[(tert-butoxycarbonyl)amino]ethyl}glycine(CAS 34046-07-6)and tert-butyl(2-aminoethyl)carbamate | LC-MS (method 3):2.32 min.MS (ESI): m/z = 495(M + H)⁺ |
| 28A | 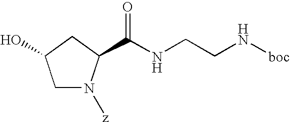 | (4R)-1-[(benzyloxy)carbonyl]-4-hydroxy-L-prolineand tert-butyl(2-aminoethyl)carbamate | LC-MS (method 2):Rₜ = 1.84 min.MS (ESI): m/z = 408(M + H)⁺ |
| 29A | 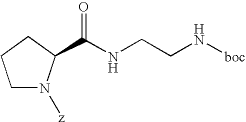 | 1-[(benzyloxy)carbonyl]-L-prolineand tert-butyl(2-aminoethyl)carbamate | LC-MS (method 3):Rₜ = 2.1 min.MS (ESI): m/z = 392(M + H)⁺ |

Examples 30A to 35A listed in the following table are prepared from the corresponding starting materials in analogy to the method of Example 2A detailed above:

| Ex. | | | |
|---|---|---|---|
| No. | Structure | Prepared from | Analytical data |
| 30A | 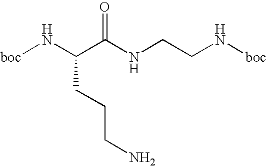 | Example 24A | MS (ESI): m/z = 375(M + H)⁺ |
| 31A | 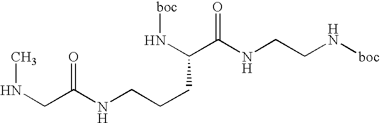 | Example 25A | MS (ESI): m/z = 445(M + H)⁺ |
| 32A | 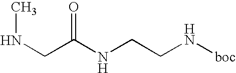 | Example 26A | MS (ESI): m/z = 232(M + H)⁺ |
| 33A | 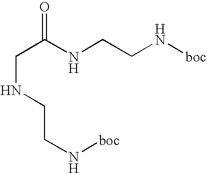 | Example 27A | MS (ESI): m/z = 361(M + H)⁺ |
| 34A | 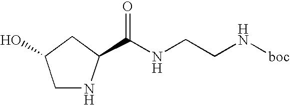 | Example 28A | MS (ESI): m/z = 274(M + H)⁺ |
| 35A | 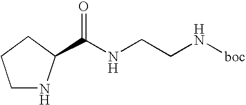 | Example 29A | MS (ESI): m/z = 258(M + H)⁺ |

### Example 36A

Benzyl{2-[{[(8S,11S,14S)-14-[(tert-butoxycarbonyl)amino]-11-{3-[(tert-butoxycarbonyl)amino]propyl}-5,17-dihydroxy-10,13-dioxo-9,12-diazatricyclo[14.3.1.1^{2,6}]henicosa-1(20),2(21),3,5,16,18-hexaen-8-yl]carbonyl}(2-hydroxyethyl)amino]ethyl}carbamate

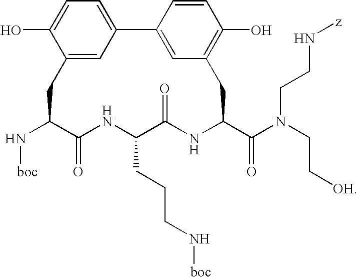

19.2 mg (0.050 mmol) of HATU and 0.010 ml (0.137 mmol) of N,N-diisopropylethylamine are added to a solution of 30 mg (0.046 mmol) of (8S,11S,14S)-14-[(tert-butoxycarbonyl)amino]-11-{3-[(tert-butoxycarbonyl)amino]propyl}5,17-dihydroxy-10,13-dioxo-9,12-diazatricyclo[14.3.1.1^{2,6}]-henicosa-1(20),2(21),3,5,16,18-hexaene-8-carboxylic acid (Example 83A from WO03/106480) in 2 ml of anhydrous DMF. After stirring at RT for 30 min, 12.7 mg (0.046 mmol) of benzyl{2-[(2-hydroxyethyl)amino]ethyl}carbamate (Example 12A) are added. The reaction mixture is stirred at RT for 15 h. The solvent is then evaporated and the residue is taken up in dichloromethane. The organic phase is washed with water, dried over magnesium sulfate and concentrated. The crude product is purified by preparative HPLC.

Yield: 8 mg (20% of theory)

LC-MS (method 2): Rₜ=2.29 min.

MS (ESI): m/z=877 (M+H)⁺.

Examples 37A to 45A listed in the following table are prepared in analogy to the method of Example 36A.

| Ex. | Precursor | | |
|---|---|---|---|
| No. | Example | Structure | Analytical data |
| 37A | Example 83Afrom WO03/106480 +13A | 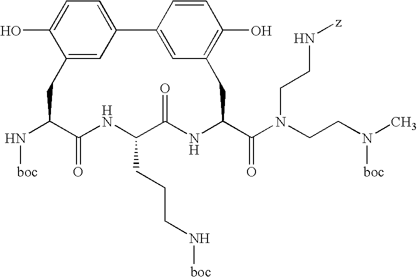 | LC-MS (method 1):Rₜ = 2.46 minMS (ESI): m/z = 990(M + H)⁺ |
| 38A | Example 83Afrom WO03/106480 +14A | 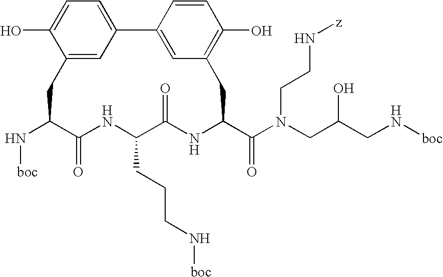 | LC-MS (method 3):Rₜ = 2.48 minMS (ESI): m/z = 1006(M + H)⁺ |
| 39A | Example 83Afrom WO03/106480 +18A | 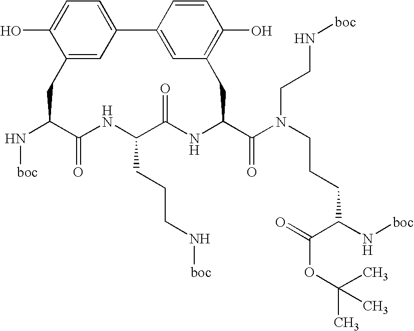 | LC-MS (method 1):Rₜ = 1.53 minMS (ESI): m/z = 1070(M + H)⁺ |
| 40A | Example 21Afrom WO03/106480 +12A | 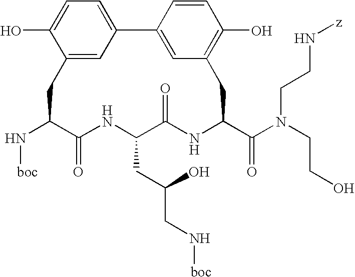 | LC-MS (method 1):Rₜ = 2.01 minMS (ESI): m/z = 893(M + H)⁺ |
| 41A | Example 83Afrom WO03/106480 +19A | 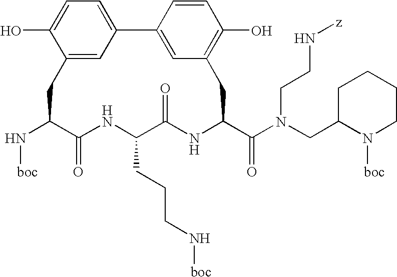 | LC-MS (method 3):Rₜ = 2.65 minMS (ESI): m/z = 1030(M + H)⁺ |
| 42A | Example 83Afrom WO03/106480 +23A | 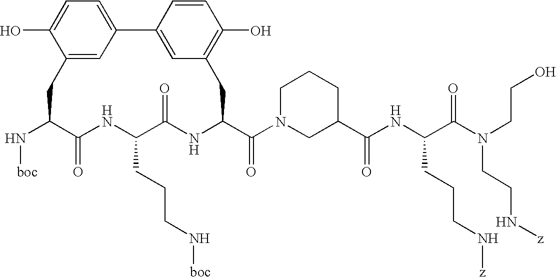 | LC-MS (method 2):Rₜ = 2.53 minMS (ESI): m/z = 1236(M + H)⁺ |
| 43A | Example 83Afrom WO03/106480 +32A | 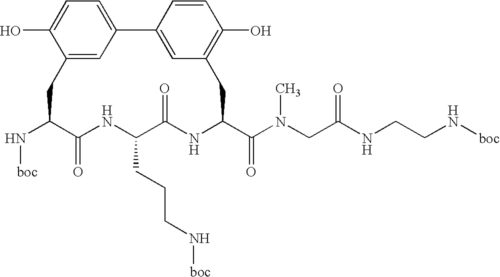 | LC-MS (method 3):Rₜ = 2.25 minMS (ESI): m/z = 870(M + H)⁺ |
| 44A | Example 83Afrom WO03/106480 +33A | 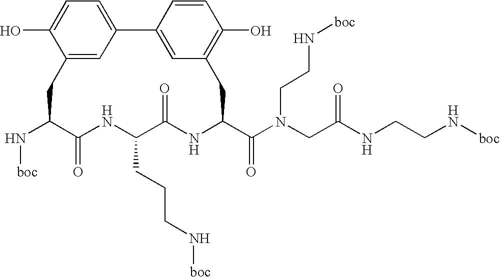 | LC-MS (method 2):Rₜ = 2.52 minMS (ESI): m/z = 999(M + H)⁺ |
| 45A | Example 83Afrom WO03/106480 +31A | 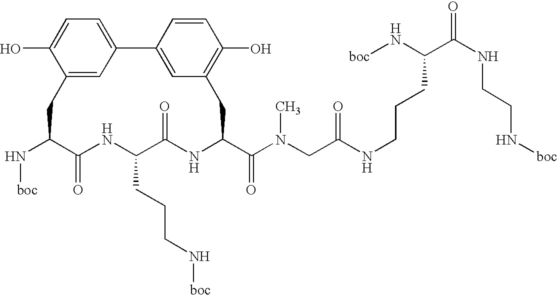 | LC-MS (method I):Rₜ = 2.20 minMS (ESI): m/z = 1084(M + H)⁺ |

### Example 46A

### 1-{[(8S,11S,14S)-14-[(tert-Butoxycarbonyl)amino]-11-{3-[(tert-butoxycarbonyl)amino]-propyl}-5,17-dihydroxy-10,13-dioxo-9,12-diazatricyclo[14.3.1.1^{2,6}]henicosa-1(20), 2(21),3,5,16,18-hexaen-8-yl]carbonyl}-L-prolyl-N⁵-(tert-butoxycarbonyl)-N-{2-[(tert-butoxycarbonyl)amino]ethyl}-L-ornithinamide

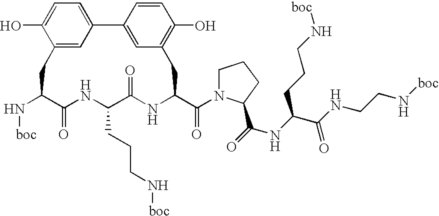

26 mg (0.050 mmol) of PyBOP and 0.020 ml (0.14 mmol) of N,N-diisopropylethylamine are added to a solution of 30 mg (0.046 mmol) of (8S,11S,14S)-14-[(tert-butoxycarbonyl)amino]-11-{3-[(tert-butoxycarbonyl)amino]propyl}-5,17-dihydroxy-10,13-dioxo-9,12-diazatricyclo[14.3.1.1^{2,6}]henicosa-1(20),2(21),3,5,16,18-hexaene-8-carboxylic acid (Example 83A of WO03/106480) in 10 ml of anhydrous DMF at 0° C. After 30 min at 0° C., 23.7 mg (0.05 mmol) of L-prolyl-N⁵-(tert-butoxycarbonyl)-N-{2-[(tert-butoxycarbonyl)amino]ethyl}-L-ornithinamide (Example 4A) are added. The reaction mixture is stirred at RT for 15 h. The solvent is then evaporated and the residue is stirred with water, collected by filtration and dried in vacuo. The crude product is purified by chromatography on Sephadex LH20 (mobile phase: methanol/acetic acid (0.25%)).

Yield: 34 mg (66% of theory)

LC-MS (method 2): Rₜ=2.49 min.

MS (ESI): m/z=1110 (M+H)⁺.

Examples 47A to 50A listed in the following table are prepared in analogy to the method of Example 46A.

| Ex. | Precursor | | |
|---|---|---|---|
| No. | Example | Structure | Analytical data |
| 47A | Example 83Afrom WO03/106480 +35A | 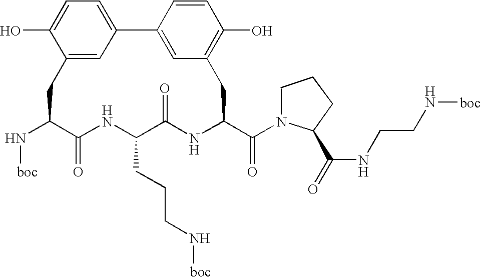 | LC-MS (method 3): Rₜ =2.26 minMS (ESI): m/z = 896(M + H)⁺ |
| 48A | Example 83Afrom WO03/106480 +10A | 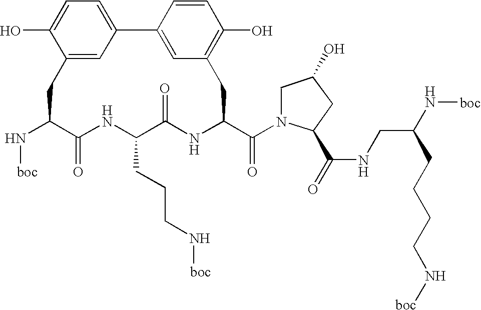 | LC-MS (method 1): Rₜ =2.29 minMS (ESI): m/z = 1083(M + H)⁺ |
| 49A | Example 21Afrom WO03/106480 +4A | 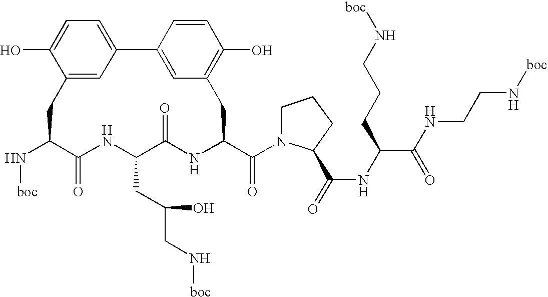 | LC-MS (method 3): Rₜ =2.21 minMS (ESI): m/z = 1126(M + H)⁺ |
| 50A | Example 83Afrom WO03/106480 +34A | 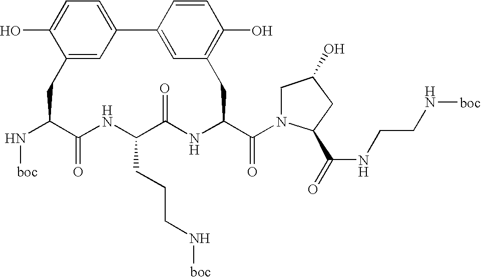 | LS-MS (method 3): Rₜ =2.15 minMS (ESI): mz/ = 912(M + H)⁺ |

### Example 51A

### tert-Butyl[2-((2-aminoethyl){[(8S,11S,14S)-14-[(tert-butoxycarbonyl)amino]-11-{3-[(tert-butoxycarbonyl)amino]propyl}-5,17-dihydroxy-10,13-dioxo-9,12-diazatricyclo-[14.3.1.1^{2,6}]henicosa-1(20),2(21),3,5,16,18-hexaen-8-yl]carbonyl}amino)ethyl]methylcarbamate hydroacetate

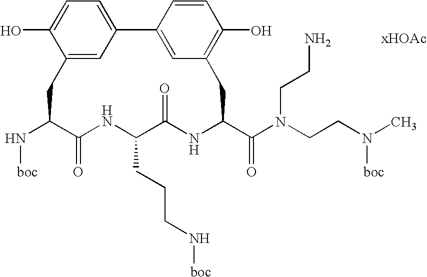

11 mg (0.01 mmol) of benzyl[2-({[(8S,11S,14S)-14-[(tert-butoxycarbonyl)amino]-11-{3-[(tert-butoxycarbonyl)amino]propyl}-5,17-dihydroxy-10,13-dioxo-9,12-diazatricyclo[14.3.1.1^{2,6}]henicosa-1(20),2(21),3,5,16,18-hexaen-8-yl]carbonyl}{2-[(tert-butoxycarbonyl)(methyl)amino]ethyl}amino)ethyl]carbamate (Example 37A) are dissolved in 4 ml of acetic acid/water (4:1). 5 mg of palladium on activated carbon (10%) are added thereto, followed by a hydrogenation under atmospheric pressure for 15 h. The reaction mixture is filtered through prewashed kieselguhr, and the filtrate is concentrated in vacuo. The crude product is reacted without further purification.

Yield: quant.

LC-MS (method 1): Rₜ=1.80 min.

MS (ESI): m/z=856 (M−HOAc+H)⁺.

Examples 52A to 56A listed in the following table are prepared in analogy to the method of Example 51A.

| Ex. | Precursor | | |
|---|---|---|---|
| No. | Example | Structure | Analytical data |
| 52A | 38A | 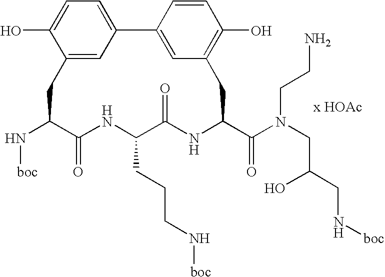 | The crude product is reactedwithout further purification. |
| 53A | 40A | 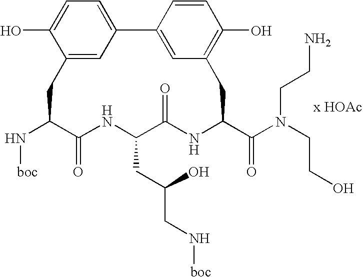 | LC-MS (method 1): Rₜ = 1.37 minMS (ESI): m/z = 759(M − HOAc + H)⁺ |
| 54A | 36A | 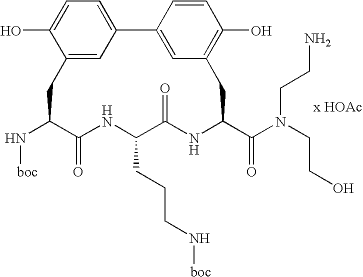 | LC-MS (method 2): Rₜ = 1.69 minMS (ESI): m/z = 742(M − HOAc + H)⁺ |
| 55A | 41A | 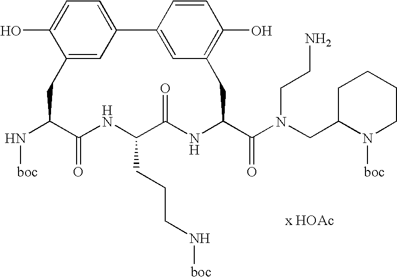 | LC-MS (method 1): Rₜ = 1.77 minMS (ESI): m/z = 896(M − HOAc + H)⁺ |
| 56A | 42A | 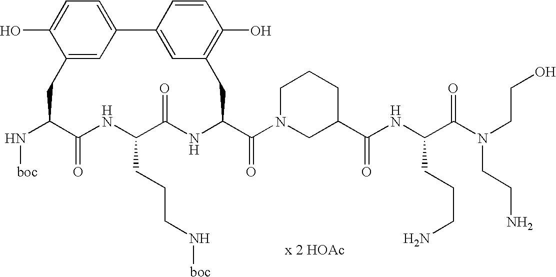 | LC-MS (method 1): Rₜ = 1.14 minMS (ESI): m/z = 968(M − 2HOAc + H)⁺ |

### EXEMPLARY EMBODIMENTS

### Example 1

### 1-{[(8S,11S,14S)-14-Amino-11-(3-aminopropyl)-5,17-dihydroxy-10,13-dioxo-9,12-diazatricyclo[14.3.1.1^{2,6}]henicosa-1(20),2(21),3,5,16,18-hexaen-8-yl]carbonyl}-L-prolyl-N-(2-aminoethyl)-L-ornithinamide tetrahydrochloride

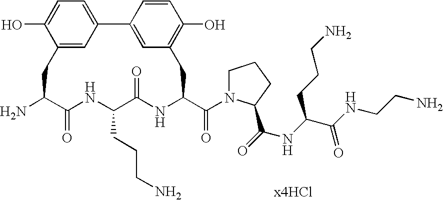

0.363 ml of a 4N hydrogen chloride solution in dioxane are added to a solution of 26.9 mg (0.024 mmol) of the compound from Example 46A in 1 ml of dioxane at 0° C. After 3 h at RT, the reaction solution is concentrated in vacuo and coevaporated with dichloromethane several times. The remaining solid is dried to constant weight under high vacuum.

Yield: 20 mg (96% of theory)

LC-MS (method 5): Rₜ=1.82 min.

MS (ESI): m/z=710 (M−4HCl+H)⁺.

¹H-NMR (400 MHz, D₂O): δ=1.26 (m_{c}, 1H), 1.55-2.1 (m, 10H), 2.27 (m_{c}, 1H), 2.75 (m_{c}, 1H), 2.9-3.2 (m, 7H), 3.3-3.8 (m, 6H), 4.25 (m_{c}, 1H), 4.44 (m_{c}, 2H), 4.7-4.9 (m, 2H, underneath D₂O), 6.85-7.0 (m, 3H), 7.27 (s, 1H), 7.34 (d, 1H), 7.4 (d, 1H).

### Example 2

### 1-{[(8S,11S,14S)-14-Amino-11-(3-aminopropyl)-5,17-dihydroxy-10,13-dioxo-9,12-diazatricyclo[14.3.1.1^{2,6}]henicosa-1(20),2(21),3,5,16,18-hexaen-8-yl]carbonyl}-L-prolyl-N-(2-aminoethyl)-L-ornithinamide tetra(hydrotrifluoroacetate)

Example 1 as tetrahydrochloride salt is converted into the tetra(hydrotrifluoroacetate) by preparative HPLC (Reprosil ODS-A, mobile phase acetonitrile/0.2% aqueous trifluoroacetic acid 5:95→95:5).

### Example 3

### (8S,11S,14S)-14-Amino-N-(2-aminoethyl)-11-(3-aminopropyl)-5,17-dihydroxy-N-[2-(methylamino)ethyl]-10,13-dioxo-9,12-diazatricyclo[14.3.1.1^{2,6}]henicosa-1(20),2(21),3,5,16,18-hexaene-8-carboxamide tetrahydrochloride

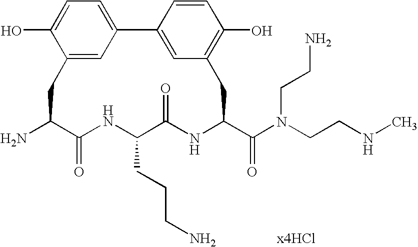

A mixture of 9 mg (0.011 mmol) of tert-butyl[2-((2-aminoethyl){[(8S,11S,14S)-14-[(tert-butoxycarbonyl)amino]-11-{3-[(tert-butoxycarbonyl)amino]propyl}-5,17-dihydroxy-10,13-dioxo-9,12-diazatricyclo[14.3.1.1^{2,6}]henicosa-1(20),2(21),3,5,16,18-hexaen-8-yl]carbonyl}amino)ethyl]methylcarbamate (Example 51A) and 1.5 ml of a 4 M hydrogen chloride solution in dioxane is stirred at RT for 20 min. The reaction solution is concentrated, coevaporated with dichloromethane several times and dried under high vacuum.

Yield: quant.

LC-MS (method 1): Rₜ=0.27 min.

MS (ESI): m/z=555 (M−4HCl+H)⁺.

¹H-NMR (400 MHz, D₂O): δ=1.60-1.90 (m, 5H), 2.73 (d, 3H), 2.86-3.11 (m, 4H), 3.23-3.38 (m, 4H), 3.50-3.95 (m, 8H), 5.09 (m, 2H), 6.96 (d, 2H), 7.01 (s, 1H), 7.33 (s, 1H), 7.40 (d, 1H), 7.46 (d, 1H).

### Example 4

### 1-{[(8S,11S,14S)-[14-Amino-11-(3-aminopropyl)-5,17-dihydroxy-10,13-dioxo-9,12-diazatricyclo[14.3.1.1^{2,6}]henicosa-[(20),2(21),3,5,16,18-hexaen-8-yl]-carbonyl}-N-(2-aminoethyl)-L-prolinamide trishydrochloride

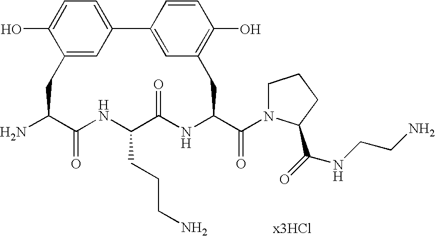

0.363 ml of a 4N hydrogen chloride solution in dioxane are added to a solution of 26.9 mg (0.024 mmol) of the compound from Example 47A in 1 ml of dioxane at 0° C. After 3 h at RT, the reaction solution is concentrated in vacuo and coevaporated with dichloromethane several times. The remaining solid is dried to constant weight under high vacuum.

Yield: 20 mg (96% of theory)

LC-MS (method 5): Rₜ=1.82 min.

MS (ESI): m/z=710 (M−3HCl+H)⁺.

¹H-NMR (400 MHz, D₂O): δ=1.26 (m_{c}, 1H), 1.5-2.1 (m, 8H), 2.26 (m_{c}, 1H), 2.76 (m_{c}, 1H), 2.9-3.2 (m, 7H), 3.3-3.6 (m, 2H), 4.37 (m_{c}, 1H), 4.45 (m_{c}, 1H), 4.7-4.9 (m, 2H, underneath D₂O), 6.85-7.0 (m, 3H), 7.27 (s, 1H), 7.34 (d, 1H), 7.4 (d, 1H).

### Example 5

### (8S,11S,14S)-14-Amino-N-{2-[(2-aminoethyl)amino]-2-oxoethyl}-11-(3-aminopropyl)-5,17-dihydroxy-N-methyl-10,13-dioxo-9,12-diazatricyclo[14.3.1.1^{2,6}]henicosa-1(20), 2(21),3,5,16,18-hexaene-8-carboxamide trishydrochloride

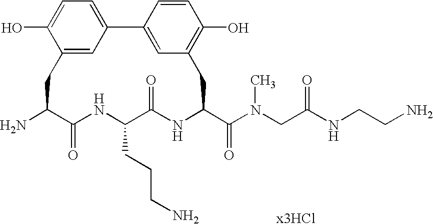

0.343 ml of a 4N hydrogen chloride solution in dioxane are added to a solution of 19.9 mg (0.023 mmol) of the compound from Example 43A and 1 ml of dioxane at 0° C. After 3 h at RT, the reaction solution is concentrated in vacuo and coevaporated with dichloromethane several times. The remaining solid is dried to constant weight under high vacuum.

Yield: 13.6 mg (88% of theory)

LC-MS (method 5): Rₜ=1.9 min.

MS (ESI): m/z=570 (M−3HCl+H)⁺.

Examples 5 to 14 listed in the following table are prepared in analogy to the method of Example 1, as hydrochloride or hydro(trifluoroacetate) salt according to the respective method of isolation.

| Example | Precursor | | |
|---|---|---|---|
| No. | Example | Structure | Analytical data |
| 6 | 54A | 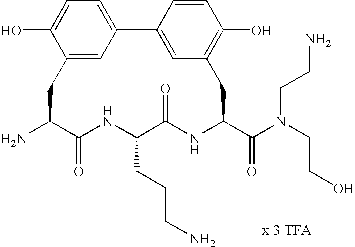 | LC-MS (method 5):Rₜ = 1.71 min.MS (ESI): m/z = 543(M − 3TFA + H)⁺.¹H-NMR (400 MHz,D₂O): δ = 1.40-2.0 (m,5H), 2.80-3.90 (m, 12H),5.18 (d, 1H), 6.93-6.96(m, 2H), 7.01 (s, 1H),7.29 (s, 1H), 7.34-7.45(m, 2H). |
| 7 | 53A | 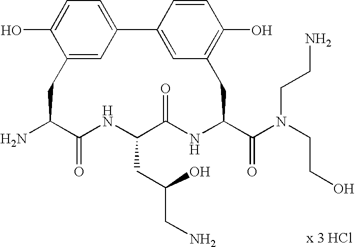 | LC-MS (method 5):Rₜ = 1.84 minMS (ESI): m/z =559 M − 3HCl + H)^{+.} |
| 8 | 52A | 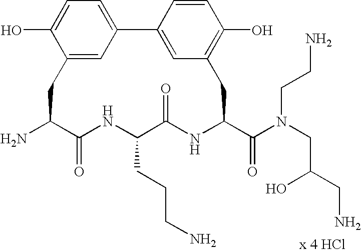 | LC-MS (method 1):Rₜ = 0.28 minMS (ESI): m/z = 572(M − 4HCl + H)⁺.¹H-NMR (400 MHz,D₂O): δ = 1.20-1.40 (m,1H), 1.60-2.0 (m, 5H),2.70-4.30 (m, 21H), 6.98-7.10 (m, 3H), 7.30-7.57(m, 3H). |
| 9 | 55A | 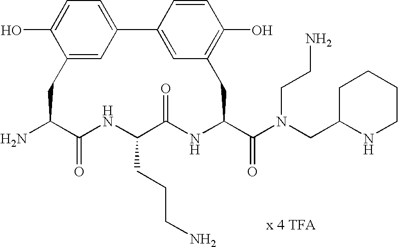 | LC-MS (method 6):Rₜ = 1.04 minMS (ESI): m/z = 596(M − 4TFA + H)⁺.¹H-NMR (400 MHz,D₂O): δ = 1.30-2.0 (m,9H), 2.70-4.00 (m, 14H),4.40 (m, 1H), 5.0 (dd_{c},1H), 6.80-6.71 (m, 3H),7.00-7.20 (m, 3H). |
| 10 | 56A | 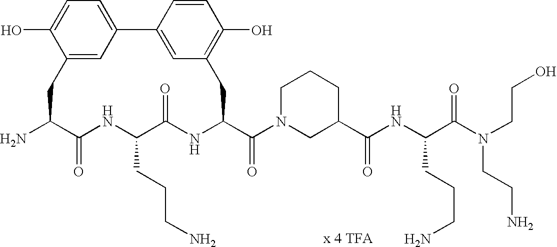 | LC-MS (method 6):Rₜ = 2.06 minMS (ESI): m/z = 768(M − 4TFA + H)⁺. |
| 11 | 39A | 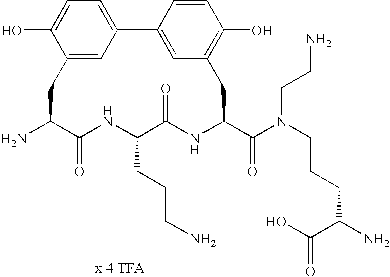 | LC-MS (method 5):Rₜ = 1.85 minMS (ESI): m/z = 614(M − 4TFA + H)⁺. |
| 12 | 48A | 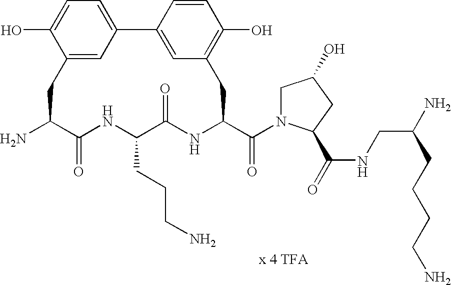 | LC-MS (method 5):Rₜ = 1.91 minMS (ESI): m/z = 683(M − 4TFA + H)⁺. |
| 13 | 49A | 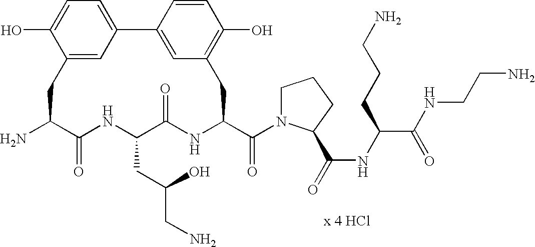 | LC-MS (method 5):Rₜ = 1.89 minMS (ESI): m/z = 726(M − 4HCl + H)⁺. |
| 14 | 44A | 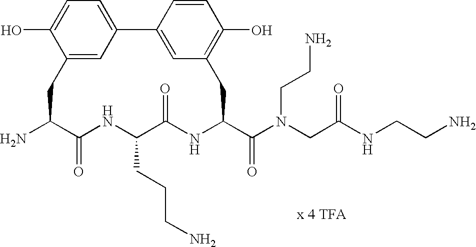 | MS (ESI): m/z = 599(M − 4TFA + H)⁺.¹H-NMR (400 MHz,D₂O): δ = 1.55-1.9 (m,4H), 2.76 (m_{c}, 1H), 2.9-3.35 (m, 10H), 3.4-3.6 (m,2H), 3.86 (m_{c}, 1H), 4.11(m_{c}, 1H), 4.34 (m_{c}, 1H),4.43 (m_{c}, 1H), 4.7-4.9 (m,1H, underneath D₂O),5.14 (m_{c}, 1H), 6.85-7.0(m, 3H), 7.2-7.45 (m,3H). |
| 15 | 45A | 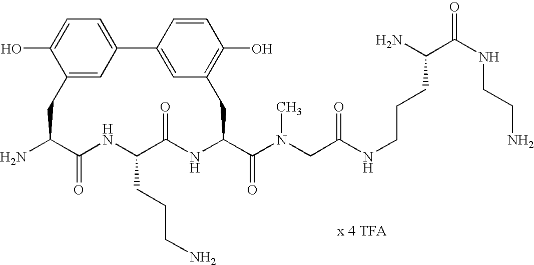 | MS (ESI): m/z = 684(M − 4TFA + H)⁺. |

### B. Assessment of the Physiological Activity

### Abbreviations Used

AMP adenosine monophosphate
ATP adenosine triphosphate
BHI medium brain heart infusion medium
CoA coenzyme A
DMSO dimethyl sulfoxide
DTT dithiothreitol
EDTA ethylenediaminetetraacetic acid
KCl potassium chloride
KH₂PO₄ potassium dihydrogen phosphate
MgSO₄ magnesium sulfate
MIC minimum inhibitory concentration
MTP microtiter plate
NaCl sodium chloride
Na₂HPO₄ disodium hydrogen phosphate
NH₄Cl ammonium chloride
NTP nucleotide triphosphate
PBS phosphate-buffered saline
PCR polymerase chain reaction
PEG polyethylene glycol
PEP phosphoenolpyruvate
Tris tris[hydroxymethyl]aminomethane
The in vitro activity of the compounds of the invention can be shown in the following assays:

In vitro Transcription-Translation with E. coli Extracts

In order to prepare an S30 extract logarithmically growing Escherichia coli MRE 600 (M. Müller; Freiburg University), are harvested, washed and employed as described for the in vitro transcription-translation test (Müller, M. and Blobel, G. Proc Natl Acad Sci USA (1984) 81, pp. 7421-7425).

1 g of cAMP (11.25 mg/ml) per 501 of reaction mix is additionally added to the reaction mix for the in vitro transcription-translation test. The test mixture amounts to 105 μl, with 5 μl of the substance to be tested being provided in 5% DMSO. 1 μg/100 μl of mixture of the plasmid pBESTL uc (Promega, Germany) is used as transcription template. After incubation at 30° C. for 60 min, 50 μl of luciferin solution (20 mM tricine, 2.67 mM MgSO₄, 0.1 mM EDTA, 33.3 mM DTT pH 7.8, 270 μM CoA, 470 μM luciferin, 530 μM ATP) are added, and the resulting bioluminescence is measured in a luminometer for 1 minute. The concentration of an inhibitor which leads to a 50% inhibition of the translation of firefly luciferase is reported as the IC₅₀.

In Vitro Transcription-Translation with S. aureus Extracts

Construction of an S. aureus luciferase Reporter Plasmid

In order to construct a reporter plasmid which can be used in an in vitro transcription-translation assay from S. aureus the plasmid pBESTluc (Promega Corporation, USA) is used. The E. coli tac promoter present in this plasmid in front of the firefly luciferase is replaced with the capA1 promoter with appropriate Shine-Dalgarno sequence from S. aureus. The primers CAPFor 5′-CGGCCAAGCTTACTC-GGATCCAGAGTTTGCAAAATATACAGGGGATTATATATAATGGAAAACAAGAAAG GAAAATAGGAGGTTTATATGGAAGACGCCA-3′ and CAPRev 5′-GTCATCGTCGGGAAGACCTG-3′ are used for this. The primer CAPFor contains the capA1 promoter, the ribosome binding site and the 5′ region of the luciferase gene. After PCR using pBESTluc as template it is possible to isolate a PCR product which contains the firefly luciferase gene with the fused capA1 promoter. This is, after restriction with ClaI and HindIII, ligated into the vector pBESTluc which has likewise been digested with ClaI and HindIII. The resulting plasmid p1a can be replicated in E. coli and can be used as template in the S. aureus in vitro transcription-translation test.

Preparation of S30 Extracts from S. aureus

Six litres of BHI medium are inoculated with a 250 ml overnight culture of an S. aureus strain and allowed to grow at 37° C. until the OD600 nm is 2-4. The cells are harvested by centrifugation and washed in 500 ml of cold buffer A (10 mM Tris acetate, pH 8.0, 14 mM magnesium acetate, 1 mM DTT, 1 M KCl). After renewed centrifugation, the cells are washed in 250 ml of cold buffer A with 50 mM KCl, and the resulting pellets are frozen at −20° C. for 60 min. The pellets are thawed on ice in 30 to 60 min and taken up to a total volume of 99 ml in buffer B (10 mM Tris acetate, pH 8.0, 20 mM magnesium acetate, 1 mM DTT, 50 mM KCl). 1.5 ml portions of lysostaphin (0.8 mg/ml) in buffer B are introduced into 3 precooled centrifuge cups and mixed with 33 ml of the cell suspension each. The samples are incubated at 37° C., shaking occasionally, for 45 to 60 min, before 150 μl of a 0.5 M DTT solution are added. The lysed cells are centrifuged at 30 000×g and 4° C. for 30 min. The cell pellet is taken up in buffer B and then centrifuged again under the same conditions, and the collected supernatants are combined. The supernatants are centrifuged again under the same conditions, and 0.25 volumes of buffer C (670 mM Tris acetate, pH 8.0, 20 mM magnesium acetate, 7 mM Na₃ phosphoenolpyruvate, 7 mM DTT, 5.5 mM ATP, 70 μM amino acids (complete from Promega), 75 μg of pyruvate kinase (Sigma, Germany))/ml are added to the upper ⅔ of the supernatant. The samples are incubated at 37° C. for 30 min. The supernatants are dialysed against 2 l of dialysis buffer (10 mM Tris acetate, pH 8.0, 14 mM magnesium acetate, 1 mM DTT, 60 mM potassium acetate) in a dialysis tube with a 3500 Da cut-off with one buffer change at 4° C. overnight. The dialysate is concentrated to a protein concentration of about 10 mg/ml by covering the dialysis tube with cold PEG 8000 powder (Sigma, Germany) at 4° C. The S30 extracts can be stored in aliquots at −70° C.

Determination of the IC₅₀ in the S. aureus In Vitro Transcription-Translation Assay

The inhibition of protein biosynthesis of the compounds can be shown in an in vitro transcription-translation assay. The assay is based on the cell-free transcription and translation of firefly luciferase using the reporter plasmid p1a as template and cell-free S30 extracts obtained from S. aureus. The activity of the resulting luciferase can be detected by luminescence measurement.

The amount of S30 extract or plasmid p1a to be employed must be tested anew for each preparation in order to ensure an optimal concentration in the assay. 3 μl of the substance to be tested, dissolved in 5% DMSO, are provided in an MTP. Then 101 of a suitably concentrated plasmid solution p1a are added. Subsequently 46 μl of a mixture of 23 μl of premix (500 mM potassium acetate, 87.5 mM Tris acetate, pH 8.0, 67.5 mM ammonium acetate, 5 mM DTT, 50 μg of folic acid/ml, 87.5 mg of PEG 8000/ml, 5 mM ATP, 1.25 mM of each NTP, 20 μM of each amino acid, 50 mM PEP (Na₃ salt), 2.5 mM cAMP, 250 μl of each E. coli tRNA/ml) and 23 μl of a suitable amount of S. aureus S30 extract are added and mixed. After incubation at 30° C. for 60 min, 50 μl of luciferin solution (20 mM tricine, 2.67 mM MgSO₄, 0.1 mM EDTA, 33.3 mM DTT pH 7.8, 270 μM CoA, 470 μM luciferin, 530 μM ATP) are, and the resulting bioluminescence is measured in a luminometer for 1 min. The concentration of an inhibitor which leads to a 50% inhibition of the translation of firefly luciferase is reported as the IC₅₀.

Determination of the Minimum Inhibitory Concentration (MIC)

The minimum inhibitory concentration (MIC) is the minimum concentration of an antibiotic with which the growth of a test microbe is inhibited over 18-24 h. The inhibitor concentration can in these cases be determined by standard microbiological methods (see, for example, The National Committee for Clinical Laboratory Standards. Methods for dilution antimicrobial susceptibility tests for bacteria that grow aerobically; approved standard-fifth edition. NCCLS document M7-A5 [ISBN 1-56238-394-9]. NCCLS, 940 West Valley Road, Suite 1400, Wayne, Pa. 19087-1898 USA, 2000). The MIC of the compounds of the invention is determined in the liquid dilution test on the 96-well microtitre plate scale. The bacterial microbes are cultivated in a minimal medium (18.5 mM Na₂HPO₄, 5.7 mM KH₂PO₄, 9.3 mM NH₄Cl, 2.8 mM MgSO₄, 17.1 mM NaCl, 0.033 μg/ml of thiamine hydrochloride, 1.2 μg/ml of nicotinic acid, 0.003 μg/ml of biotin, 1% glucose, 25 μg/ml of each proteinogenic amino acid with the exception of phenylalanine; [H.-P. Kroll; unpublished]) with addition of 0.4% BH broth (test medium). In the case of Enterococcus faecium L4001, heat-inactivated fetal calf serum (FCS; GibcoBRL, Germany) is added to the test medium in a final concentration of 10%. Overnight cultures of the test microbes are diluted to an OD₅₇₈ of 0.001 (to 0.01 in the case of enterococci) in fresh test medium, and incubated 1:1 with dilutions of the test substances (1:2 dilution steps) in test medium (200 μl final volume). The cultures are incubated at 37° C. for 18-24 hours; enterococci in the presence of 5% CO₂

The lowest substance concentration in each case at which no visible bacterial growth occurs any more is defined as the MIC.

Alternative Method for Determining the Minimum Inhibitory Concentration (MIC)

The minimum inhibitory concentration (MIC) is the minimum concentration of an antibiotic with which the growth of a test microbe is inhibited over 18-24 h. The inhibitor concentration can in these cases be determined by standard microbiological methods with modified medium in an agar dilution test (see, for example, The National Committee for Clinical Laboratory Standards. Methods for dilution antimicrobial susceptibility tests for bacteria that grow aerobically; approved standard-fifth edition. NCCLS document M7-A5 [ISBN 1-56238-394-9]. NCCLS, 940 West Valley Road, Suite 1400, Wayne, Pa. 19087-1898 USA, 2000). The bacterial microbes are cultivated on 1.5% agar plates which contain 20% defibrinated horse blood. The test microbes, which are incubated overnight on Columbia blood agar plates (Becton-Dickinson), are diluted in PBS, adjusted to a microbe count of about 5×10⁵ microbes/ml and placed dropwise (1-3 μl) on test plates. The test substances comprise different dilutions of the test substances (1:2 dilution steps). The cultures are incubated at 37° C. in the presence of 5% CO₂ for 18-24 hours.

The lowest substance concentration in each case at which no visible bacterial growth occurred any more is defined as the MIC and is reported in μg/ml.

TABLE A
(with comparative example biphenomycin B)

| | MIC | MIC | MIC | IC₅₀ |
|---|---|---|---|---|
| | S. aureus | S. aureus | E. faecium | S. aureus 133 |
| Ex. No. | 133 | T17 | L4001 | Translation |
| 1 | 1.0 | 4.0 | 2.0 | 0.7 |
| 3 | 2.0 | 4.0 | 8.0 | 0.8 |
| 6 | 2.0 | 4.0 | 4.0 | 0.7 |
| 8 | 1.0 | 4.0 | 16.0 | 0.25 |
| 9 | 4.0 | 2.0 | 32 | 0.33 |
| Biphenomycin B | <0.03 | >32 | 0.5 | 1.5 |

Concentration data: MIC in μg/ml; IC₅₀ in μM.

Systemic Infection with S. aureus 133

The suitability of the compounds of the invention for the treatment of bacterial infections can be shown in various animal models. For this purpose, the animals are generally infected with a suitable virulent microbe and then treated with the compound to be tested, which is present in a formulation which is adapted to the particular therapy model. Specifically the suitability of the compounds of the invention for the treatment of bacterial infections can be demonstrated in a mouse sepsis model after infection with S. aureus.

For this purpose, S. aureus 133 cells are cultured overnight in BH broth (Oxoid, Germany). The overnight culture was diluted 1:100 in fresh BH broth and expanded for 3 hours. The bacteria which are in the logarithmic phase of growth are centrifuged and washed twice with buffered physiological saline solution. A cell suspension in saline solution with an extinction of 50 units is then adjusted in a photometer (Dr Lange LP 2W). After a dilution step (1:15), this suspension is mixed 1:1 with a 10% mucine suspension. 0.2 ml of this infection solution is administered i.p. per 20 g of mouse. This corresponds to a cell count of about 1-2×10⁶ microbes/mouse. The i.v. therapy takes place 30 minutes after the infection. Female CFW1 mice are used for the infection experiment. The survival of the animals is recorded for 6 days. The animal model is adjusted so that untreated animals die within 24 h after the infection. It was possible to demonstrate in this model a therapeutic effect of ED₁₀₀=1.25 mg/kg for the compound of Example 2.

Determination of Spontaneous Resistance Rates to S. aureus

The spontaneous resistance rates for the compounds of the invention are determined as follows: the bacterial microbes are cultivated in 30 ml of a minimal medium (18.5 mM Na₂HPO₄, 5.7 mM KH₂PO₄, 9.3 mM NH₄Cl, 2.8 mM MgSO₄, 17.1 mM NaCl, 0.033 μg/ml of thiamine hydrochloride, 1.2 μg/ml of nicotinic acid, 0.003 μg/ml of biotin, 1% glucose, 25 μg/ml of each proteinogenic amino acid with the addition of 0.4% BH broth) at 37° C. overnight, centrifuged at 6000×g for 10 min and resuspended in 2 ml of a phosphate-buffered physiological NaCl solution (about 2×10⁹ microbes/ml). 100 μl of this cell suspension, and 1:10 and 1:100 dilutions respectively, are plated out on predried agar plates (1.5% agar, 20% defibrinated horse blood, or 1.5% agar, 20% bovine serum in 1/10 Müller-Hinton medium diluted with PBS respectively) which contain the compound of the invention to be tested in a concentration equivalent to 5×MIC or 10×MIC respectively, and incubated at 37° C. for 48 h. The resulting colonies (cfu) are counted.

Isolation of the Biphenomycin-Resistant S. aureus Strains RN4220Bi^{R} and T17

The S. aureus strain RN4220Bi^{R} is isolated in vitro. For this purpose, 100 μl portions of an S. aureus RN4220 cell suspension (about 1.2×10⁸ cfu/ml) are plated out on an antibiotic-free agar plate (18.5 mM Na₂HPO₄, 5.7 mM KH₂PO₄, 9.3 mM NH₄Cl, 2.8 mM MgSO₄, 17.1 mM NaCl, 0.033 μg/ml of thiamine hydrochloride, 1.2 μg/ml of nicotinic acid, 0.003 μg/ml of biotin, 1% glucose, 25 μg/ml of each proteinogenic amino acid with the addition of 0.4% BH broth and 1% agarose) and on an agar plate containing 2 μg/ml biphenomycin B (10×MIC), and incubated at 37° C. overnight. Whereas about 1×10⁷ cells grow on the antibiotic-free plate, about 100 colonies grow on the antibiotic-containing plate, corresponding to a resistance rate of 1×10⁵. Some of the colonies grown on the antibiotic-containing plate are tested for the biphenomycin B MIC. One colony with a MIC of >50 μM is selected for further use, and the strain is referred to as RN4220Bi^{R}.

The S. aureus strain T17 is isolated in vivo. CFW1 mice are infected intraperitoneally with 4×10⁷ S. aureus 133 cells per mouse. 0.5 h after the infection, the animals are treated intravenously with 50 mg/kg biphenomycin B. The kidneys are removed from the surviving animals on day 3 after the infection. After homogenization of the organs, the homogenates are plated out as described for RN4220Bi^{R} on antibiotic-free and antibiotic-containing agar plates and incubated at 37° C. overnight. About half the colonies isolated from the kidney show growth on the antibiotic-containing plates (2.2×10⁶ colonies), demonstrating the accumulation of biphenomycin B-resistant S. aureus cells in the kidney of the treated animals. About 20 of these colonies are tested for the biphenomycin B MIC, and a colony with a MIC of >50 μM is selected for further cultivation, and the strain is referred to as T17.

### C. Exemplary Embodiments of Pharmaceutical Compositions

The compounds of the invention can be converted into pharmaceutical preparations in the following way:

Solution which can be Administered Intravenously:

Composition:

1 mg of the compound of Example 1, 15 g of polyethylene glycol 400 and 250 g of water for injections.

Preparation:

The compound of the invention is dissolved together with polyethylene glycol 400 in the water with stirring. The solution is sterilized by filtration (pore diameter 0.22 μm) and dispensed under aseptic conditions into heat-sterilized infusion bottles. The latter are closed with infusion stoppers and crimped caps.

What is claimed, is:

1. A compound of formula 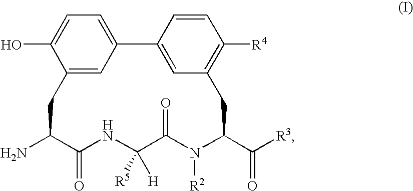
in which
R⁵ represents a group of formula 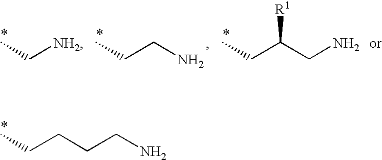
whereby
* is the linkage site to the carbon atom,
R¹ represents hydrogen or hydroxy,
R² represents hydrogen, methyl or ethyl,
R³ represents a group of formula 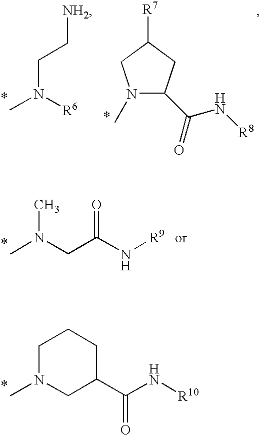
whereby
* is the linkage site to the nitrogen atom,
R⁶ represents a group of formula 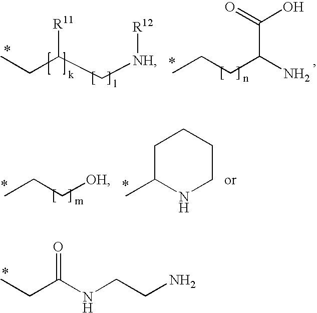
in which
* is the linkage site to the nitrogen atom,
R¹¹ represents hydrogen, amino or hydroxy,
R¹² represents hydrogen or methyl,
k is a number 0 or 1,
l is a number 1, 2, 3 or 4,
and
m and n independently of one another are a number 1, 2 or 3,
R⁷ represents hydrogen, amino or hydroxy,
R⁸, R⁹ and R¹⁰ independently of one another represent a group of formula 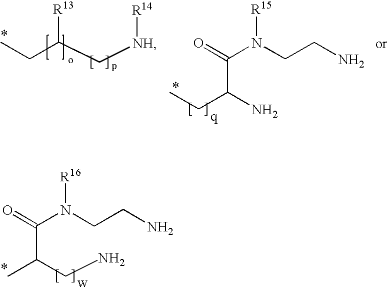
in which
* is the linkage site to the nitrogen atom
R¹³ represents hydrogen, amino or hydroxy,
R¹⁴ represents hydrogen or methyl,
R¹⁵ and R¹⁶ independently of one another represent hydrogen, aminoethyl or hydroxyethyl,
o is a number 0 or 1,
p, q and w independently of one another are a number 1, 2, 3 or 4,
R⁴ represents hydrogen, hydroxy, halogen, amino or methyl,
or one of its salts, its solvates or the solvates of its salts.

2. The compound of claim 1, corresponding to formula 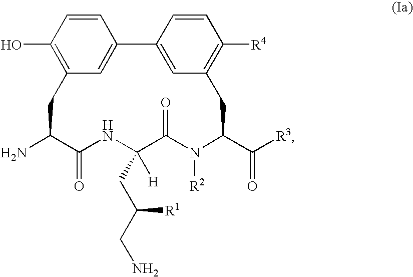
in which
R¹ represents hydrogen or hydroxy,
R² represents hydrogen, methyl or ethyl,
R³ is as defined above,
R⁴ represents hydrogen, hydroxy, halogen, amino or methyl,
or one of its salts, its solvates or the solvates of its salts.

3. The compound of claim 1, wherein
R² represents hydrogen.

4. The compound of claim 1, wherein
R⁴ represents hydrogen, hydroxy, chlorine or methyl.

5. The compound of claim 1, wherein
R⁵ represents a group of formula 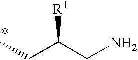
whereby
* is the linkage site to the carbon atom,
R¹ represents hydrogen or hydroxy,
R² represents hydrogen,
R³ represents a group of formula 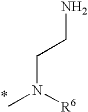
whereby
* is the linkage site to the nitrogen atom,
R⁶ represents a group of formula 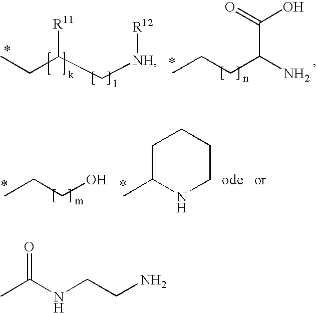
in which
* is the linkage site to the nitrogen atom,
R¹¹ represents hydrogen, amino or hydroxy,
R¹² represents hydrogen or methyl,
k is a number 0 or 1,
l is a number 1, 2, 3 or 4,
and
m and n independently of one another are a number 1, 2 or 3,
R⁴ represents hydroxy,
or one of its salts, its solvates or the solvates of its salts.

6. The compound of claim 1, wherein
R⁵ represents a group of formula 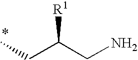
whereby
* is the linkage site to the carbon atom,
R¹ represents hydrogen or hydroxy,
R² represents hydrogen,
R³ represents a group of formula 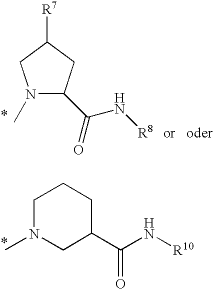
whereby
* is the linkage site to the nitrogen atom,
R⁷ represents hydrogen, amino or hydroxy,
R⁸ and R¹⁰ independently of one another represent a group of formula 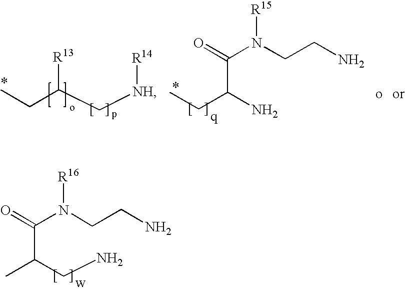
in which
* is the linkage site to the nitrogen atom,
R¹³ represents hydrogen, amino or hydroxy,
R¹⁴ represents hydrogen or methyl,
R¹⁵ and R¹⁶ independently of one another represent hydrogen, aminoethyl or hydroxyethyl,
o is a number 0 or 1,
p, q and w independently of one another are a number 1, 2, 3 or 4,
R⁴ represents hydroxy,
or one of its salts, its solvates or the solvates of its salts.

7. The compound of claim 1, wherein
R⁵ represents a group of formula 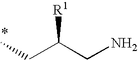
whereby
* is the linkage site to the carbon atom,
R¹ represents hydrogen or hydroxy,
R² represents hydrogen,
R³ represents a group of formula 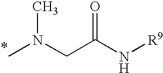
whereby
* is the linkage site to the nitrogen atom,
R⁹ represents a group of formula 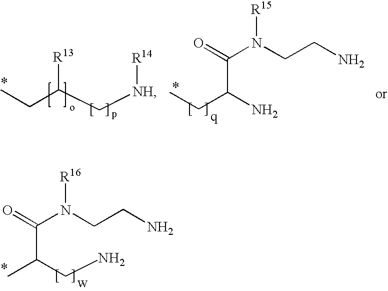
in which
* is the linkage site to the nitrogen atom,
R¹³ represents hydrogen, amino or hydroxy,
R¹⁴ represents hydrogen or methyl,
R¹⁵ and R¹⁶ independently of one another represent hydrogen, aminoethyl or hydroxyethyl,
o is a number 0 or 1,
p, q and w independently of one another are a number 1, 2, 3 or 4,
R⁴ represents hydroxy,
or one of its salts, its solvates or the solvates of its salts.

8. A method for preparing a compound of formula (I) of claim 1 or one of its salts, solvates or solvates of its salts, wherein
[A] a compound of formula 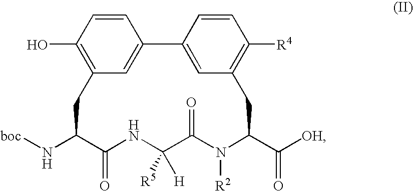
in which R², R⁴ and R⁵ have the meaning indicated in claim 1, and boc represents tert-butoxycarbonyl,
is reacted in a two-stage process firstly in the presence of one or more dehydrating reagents with a compound of formula
HR³ (III)
in which R³ has the meaning indicated in claim 1,
and subsequently with an acid, by hydrogenolysis, or with an acid and by hydrogenolysis,
or
[B] a compound of formula 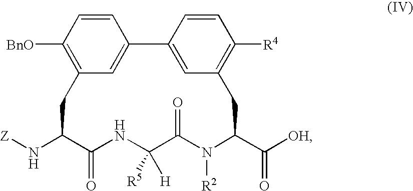
in which R², R⁴ and R⁵ have the meaning indicated in claim 1 and Z represents benzyloxycarbonyl,
is reacted in a two-stage process firstly in the presence of one or more dehydrating reagents with a compound of formula
HR³ (III)
in which R³ has the meaning indicated in claim 1,
and subsequently with an acid or by hydrogenolysis.

9. A method for preparing a compound of formula (I) of claim 1 or one of its solvates, wherein a salt of said compound or a solvate of a salt of said compound is converted into said compound by chromatography with addition of a base.

10. The compound of claim 1 for the treatment, prophylaxis or treatment and prophylaxis of diseases.

11. A method for the production of a medicament for the treatment, prophylaxis or treatment and prophylaxis of diseases, using a compound of claim 1.

12. A method for the production of a medicament for the treatment, prophylaxis or treatment and prophylaxis of bacterial diseases, using a compound of claim 1.

13. A medicament comprising at least one compound of claim 1 in combination with at least one inert, non-toxic, pharmaceutically acceptable excipient.

14. The medicament of claim 13 for the treatment, prophylaxis or treatment and prophylaxis of bacterial infections.

15. A method for controlling bacterial infections in humans and animals by administering an antibacterially effective amount of at least one compound of claim 1.

16. A method for controlling bacterial infections in humans and animals by administering an antibacterially effective amount of a medicament of claim 13.
